(19) United States
(12) Patent Application Publication
(10) Pub. No.: US 2025/0288428 A1
(43) Pub. Date: Sep. 18, 2025

(54) MODULAR SPINAL IMPLANT DEVICE

(71) Applicant: Spinal Elements, Inc., Carlsbad, CA (US)

(72) Inventors: Benjamin Meyer, Carlsbad, CA (US); Darren Reimers, Oceanside, CA (US); Markanthony Flores, Chula Vista, CA (US); Matthew Copp, Portage, MI (US)

(21) Appl. No.: 19/075,582

(22) Filed: Mar. 10, 2025

Related U.S. Application Data
(60) Provisional application No. 63/564,202, filed on Mar. 12, 2024.

(51) Int. Cl. A61F 2/44; A61F 2/30

(52) U.S. Cl. CPC A61F 2/447; A61F 2/30771; A61F 2002/30003; A61F 2002/30364; A61F 2002/30522; A61F 2002/3092; A61F 2002/3093; A61F 2002/30934

(57) ABSTRACT

In some embodiments, a spinal implant device is provided. The spinal implant device can include a shell. The shell can include a movable lid forming an upper surface of the shell. In some embodiments, the movable lid is configured to rotate and translate. The spinal implant device can include an insert configured to couple with the shell. In some embodiments, a spinal implant device is provided. The spinal implant device can include a body, a movable lid configured to rotate relative to the body, and an endplate. The body, the movable lid, and the endplate can form a cavity configured to be packed with material

### CROSS-REFERENCE TO RELATED APPLICATIONS

This application claims priority benefit to U.S. Provisional Patent Application No. 63/564,202, filed Mar. 12, 2024, the entirety of which is hereby incorporated by reference herein.

### BACKGROUND

### Field

Some embodiments described herein relate generally to systems and methods for stabilizing bone, for example, stabilizing vertebrae by fusing adjacent vertebrae. Some embodiments relate to spinal implant devices, for example spinal implant devices with a modular design.

### Description of the Related Art

Advancing age, as well as injury, can lead to degenerative changes in the bones, discs, joints, and ligaments of the spine. These degenerative changes can produce pain and spinal instability. Under certain circumstances, spinal fusion can alleviate these degenerative changes. Spinal fusion is a surgical technique in which two or more vertebrae of the spinal column are fused together to reduce or limit the motion between the vertebrae. Spinal fusion is used to treat various conditions including fracture, scoliosis, and spondylolisthesis. Spinal fusion with discectomy is used to treat herniation of the discs by removal of the affected disc and fusion of the adjacent vertebrae. There are several procedures available to patients with degenerative spine conditions.

In some methods, a spinal implant device is inserted into the disc space to promote bone growth between adjacent vertebrae. The spinal implant device is positioned between adjacent vertebrae in the disc space. In some methods, the spinal implant device can be secured to one or more vertebrae by bone screws or other similar fasteners inserted through holes in the spinal implant device. The size of the spinal implant device is typically selected such that the spinal implant device forces the vertebrae apart to cause tensing of the vertebral annulus and other soft tissue structures surrounding the joint space. Tensing the soft tissues surrounding the joint space can result in the vertebrae exerting compressive forces on the spinal implant device to maintain the spinal implant device in place.

The current standard of care to address the degenerative changes of the spine is to fuse vertebrae. The relative motion between the two adjacent vertebrae is limited or reduced in some methods of use.

### SUMMARY

In some embodiments, a spinal implant device is provided. The spinal implant device can include a shell. The shell can include a distal end and a proximal end. The shell can include a lower wall forming a lower surface of the shell. The shell can include a movable lid forming an upper surface of the shell. In some embodiments, the movable lid is configured to rotate and translate. The spinal implant device can include an insert configured to couple with the shell.

In some embodiments, the movable lid is configured to rotate up to 180 degrees. In some embodiments, the movable lid is configured to translate between 0 mm and 6 mm. In some embodiments, the spinal implant device can include a plurality of inserts. In some embodiments, the plurality of inserts comprises different lordosis. In some embodiments, the plurality of inserts comprises different heights. In some embodiments, the plurality of inserts comprises different materials. In some embodiments, the insert is configured to be lowered toward the lower wall to couple with the shell. In some embodiments, the insert and the shell form a cavity configured to be packed with graft material. In some embodiments, the insert comprises a retention feature and the movable lid comprises a complementary retention feature. In some embodiments, the insert comprises a first insert opening and the lower wall comprises a first opening, wherein the first insert opening and the first opening are aligned along a trajectory for the insertion of a first fastener. In some embodiments, the insert comprises a second insert opening and the movable lid comprises a second opening, wherein the second insert opening and the second opening are aligned along a trajectory for the insertion of a second fastener. In some embodiments, the insert comprises a third insert opening and the lower wall comprises a third opening, wherein the third insert opening and the third opening are aligned along a trajectory for the insertion of a third fastener. In some embodiments, the insert comprises a first insert opening and the movable lid comprises a first opening, wherein the first insert opening and the first opening are aligned along a trajectory for the insertion of a first fastener. In some embodiments, the insert comprises a second insert opening and the lower wall comprises a second opening, wherein the second insert opening and the second opening are aligned along a trajectory for the insertion of a second fastener. In some embodiments, the insert comprises a third insert opening and the movable lid comprises a third opening, wherein the third insert opening and the third opening are aligned along a trajectory for the insertion of a third fastener. In some embodiments, the shell is 3D printed as one piece. In some embodiments, the shell comprises a porous or network surface. In some embodiments, the movable lid comprises a pin joint.

In some embodiments, a method is provided. The method can include providing a shell. The shell can include a distal end and a proximal end. The shell can include a lower wall forming a lower surface of the shell. The shell can include a movable lid. The method can include opening the movable lid. In some embodiments, the movable lid rotates and translates. The method can include lowering an insert toward the lower wall to couple with the shell.

In some embodiments, the method can include packing a cavity formed by the insert and the shell with graft material. In some embodiments, the method can include selecting the insert from a plurality of inserts. In some embodiments, the method can include closing the movable lid.

In some embodiments, a spinal implant device is provided. The spinal implant device can include a body. The spinal implant device can include a movable lid configured to rotate relative to the body. The spinal implant device can include an endplate. In some embodiments, the body, the movable lid and the endplate form a cavity configured to be packed with material. In some embodiments, the spinal implant device comprises a pin, wherein the movable lid is configured to rotate relative to the pin. In some embodiments, the pin comprises titanium. In some embodiments, the movable lid and the endplate are separate components configured to be assembled onto the body. In some embodiments, the movable lid is printed as a separate piece. In some embodiments, the movable lid and the body comprise a printed assembly. In some embodiments, the endplate is printed as a separate piece. In some embodiments, the endplate and the body comprise a printed assembly. In some embodiments, the endplate and the body are coupled with one or more pins. In some embodiments, the movable lid comprises titanium. In some embodiments, the body comprises PEEK. In some embodiments, the endplate comprises titanium. In some embodiments, an upper wall comprises titanium and PEEK.

### BRIEF DESCRIPTION

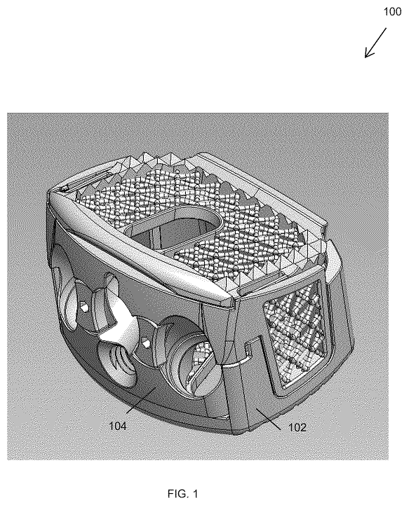

FIG. 1 is a perspective view of an embodiment of a spinal implant device with a movable lid shown in a closed position.

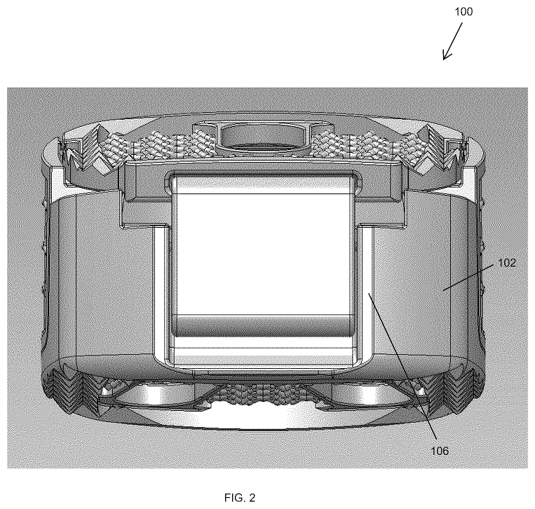

FIG. 2 is a distal view of the spinal implant device of FIG. 1.

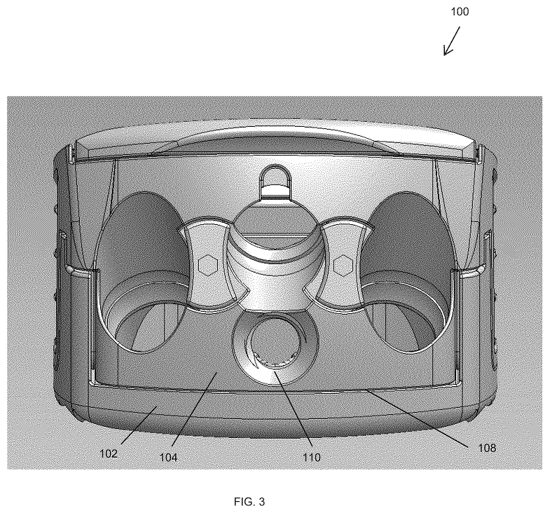

FIG. 3 is a proximal view of the spinal implant device of FIG. 1.

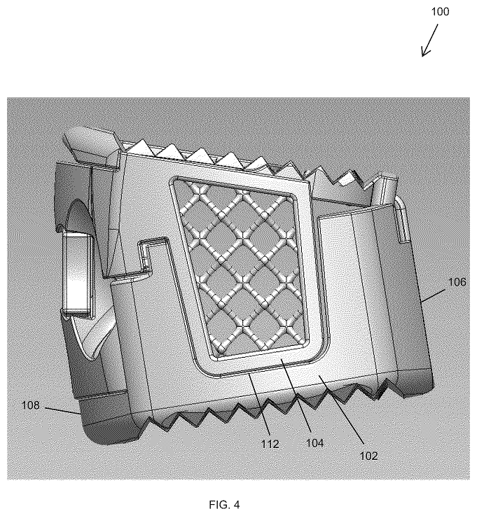

FIG. 4 is a side view of the spinal implant device of FIG. 1.

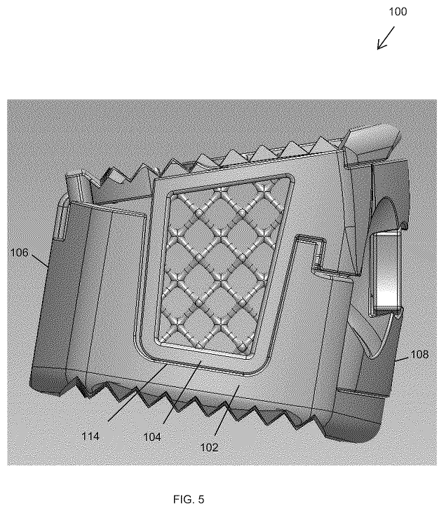

FIG. 5 is another side view of the spinal implant device of FIG. 1.

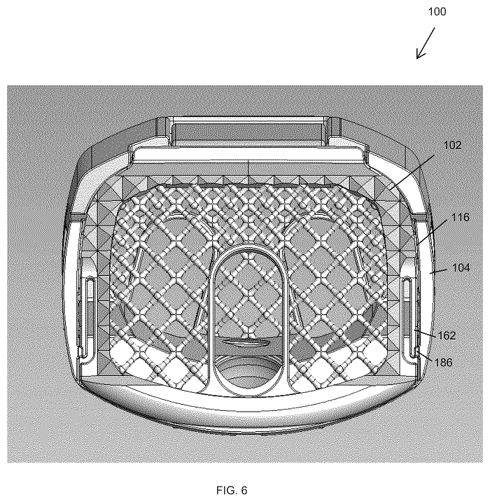

FIG. 6 is a top view of the spinal implant device of FIG. 1.

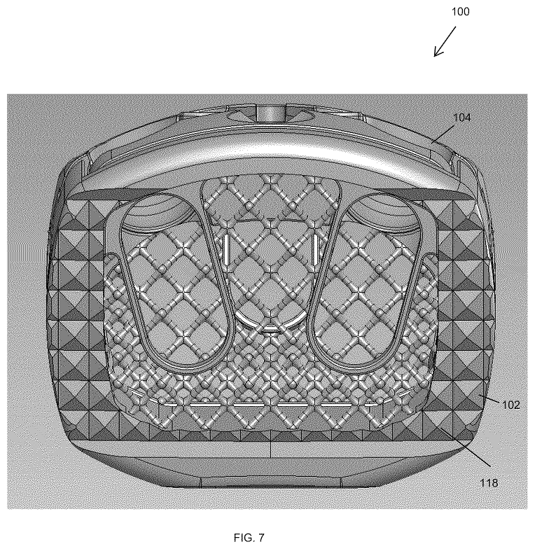

FIG. 7 is a bottom view of the spinal implant device of FIG. 1.

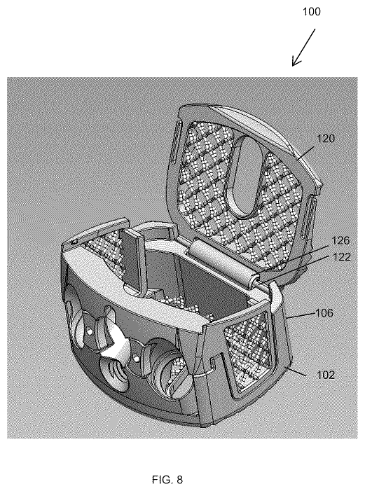

FIG. 8 is a top perspective view of the spinal implant device of FIG. 1 with the movable lid shown in a rotated position.

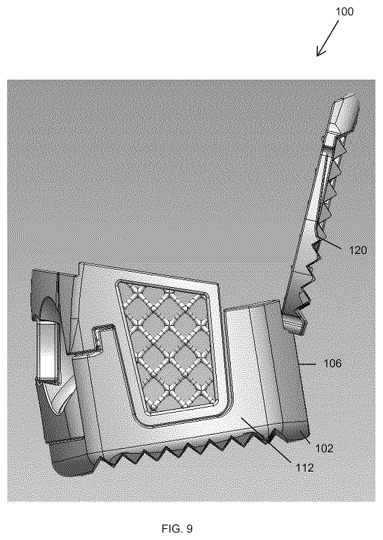

FIG. 9 is a side view of the spinal implant device of FIG. 1 with the movable lid shown in a rotated position.

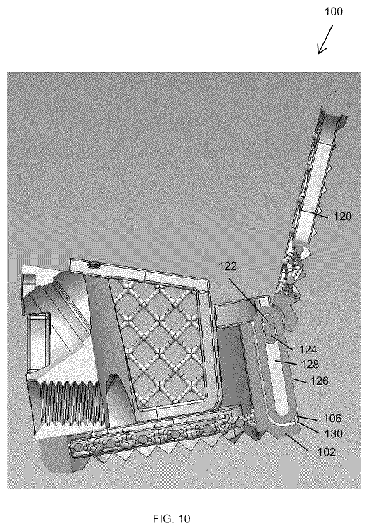

FIG. 10 is a cross-sectional view of the spinal implant device of FIG. 1 with the movable lid shown in a rotated position.

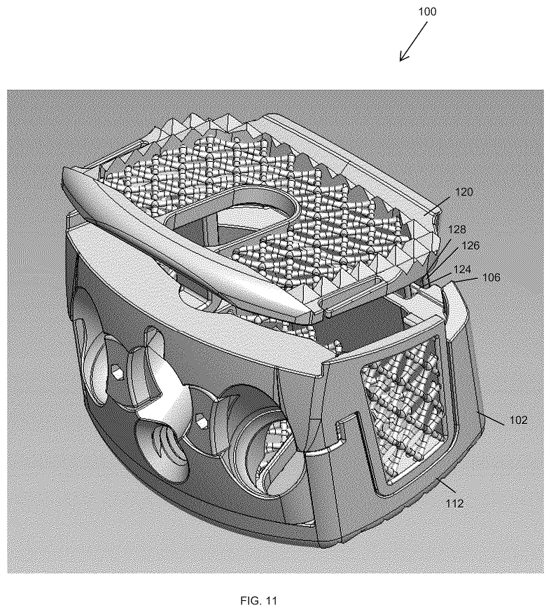

FIG. 11 is a top perspective view of the spinal implant device of FIG. 1 with the movable lid shown in a translated position.

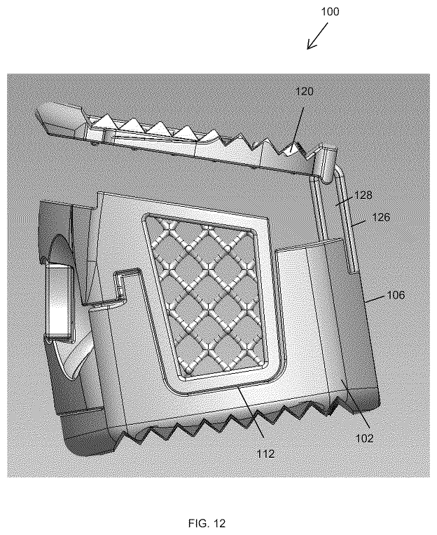

FIG. 12 is a side view of the spinal implant device of FIG. 1 with the movable lid shown in a translated position.

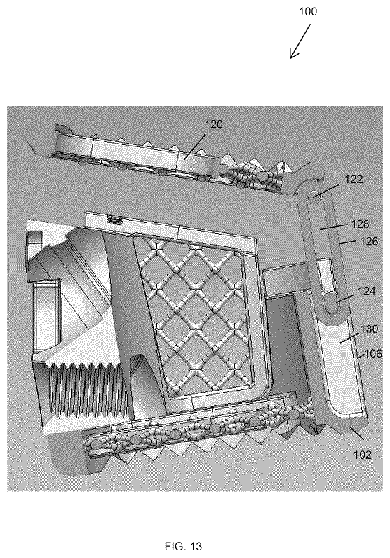

FIG. 13 is a cross-sectional view of the spinal implant device of FIG. 1 with the movable lid shown in a translated position.

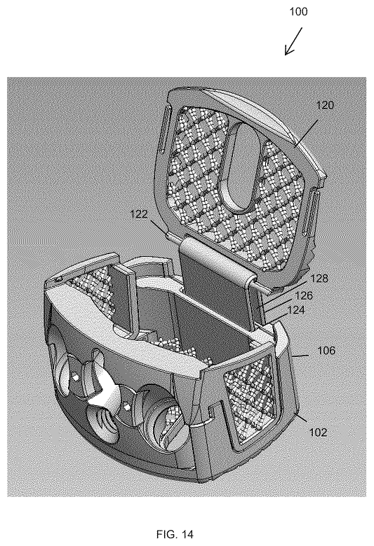

FIG. 14 is a top perspective view of the spinal implant device of FIG. 1 with the movable lid shown in a rotated and translated position.

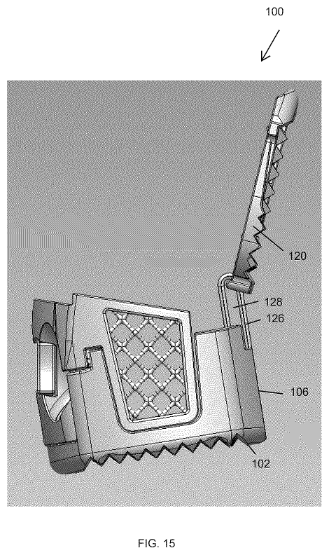

FIG. 15 is a side view of the spinal implant device of FIG. 1 with the movable lid shown in a rotated and translated position.

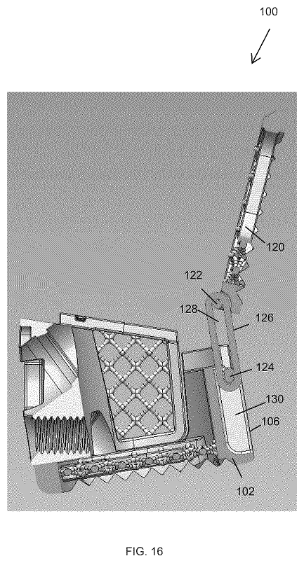

FIG. 16 is a cross-sectional view of the spinal implant device of FIG. 1 with the movable lid shown in a rotated and translated position.

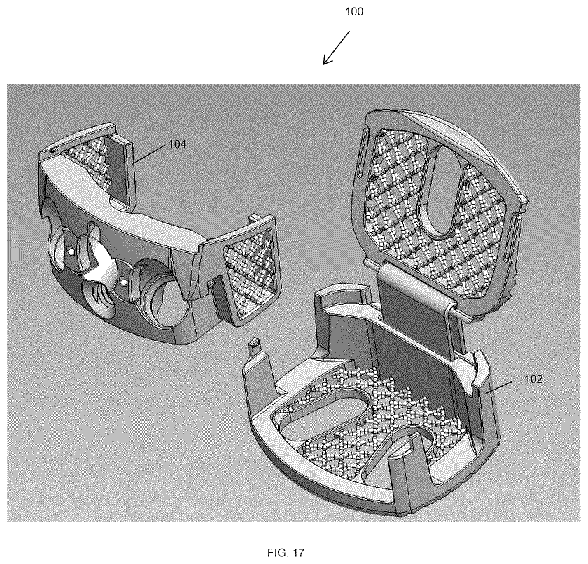

FIG. 17 is an exploded perspective view of the spinal implant device of FIG. 1.

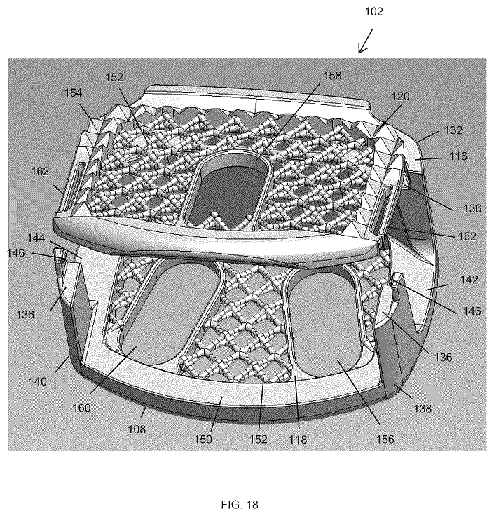

FIG. 18 is a top perspective view of a shell of FIG. 1.

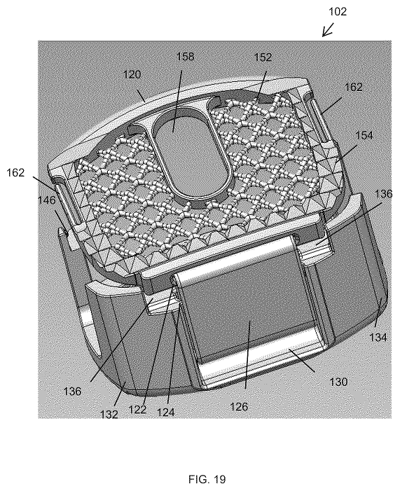

FIG. 19 is another top perspective view of the shell of FIG. 1.

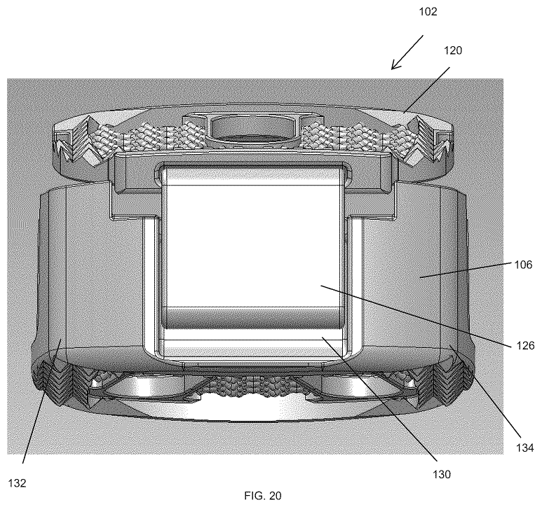

FIG. 20 is a distal view of the shell of FIG. 1.

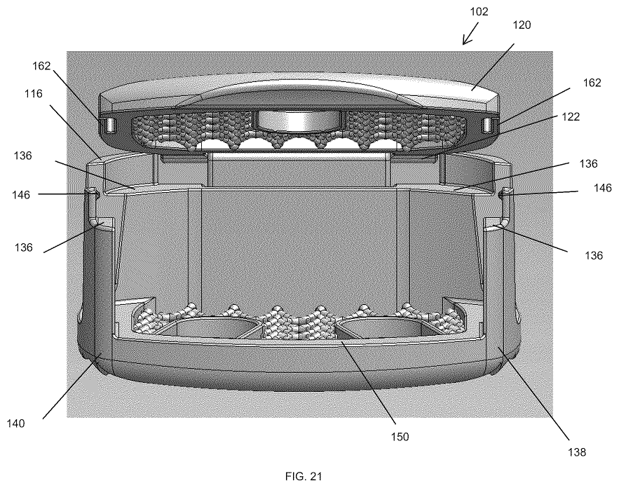

FIG. 21 is a proximal view of the shell of FIG. 1.

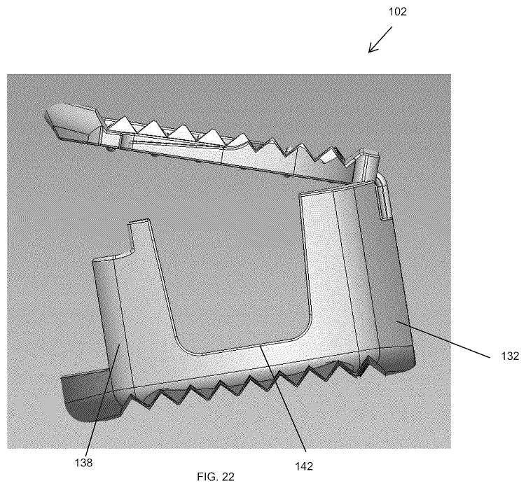

FIG. 22 is a side view of the shell of FIG. 1.

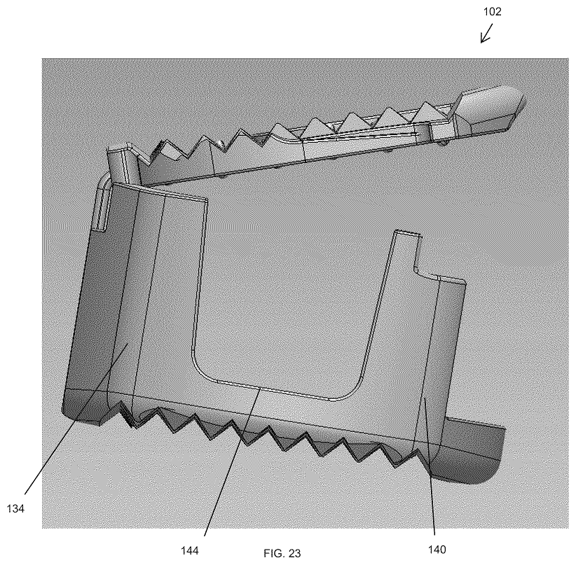

FIG. 23 is another side view of the shell of FIG. 1.

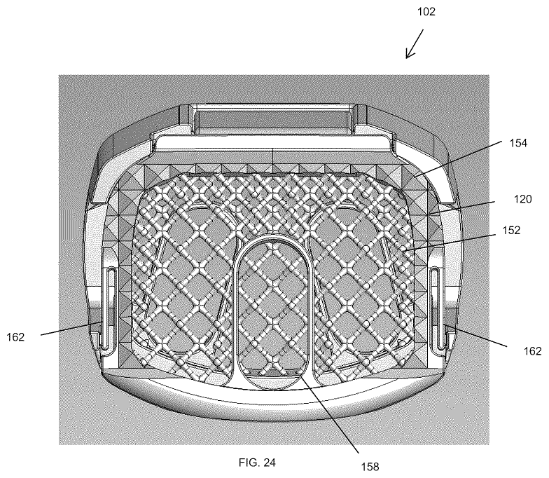

FIG. 24 is a top view of the shell of FIG. 1.

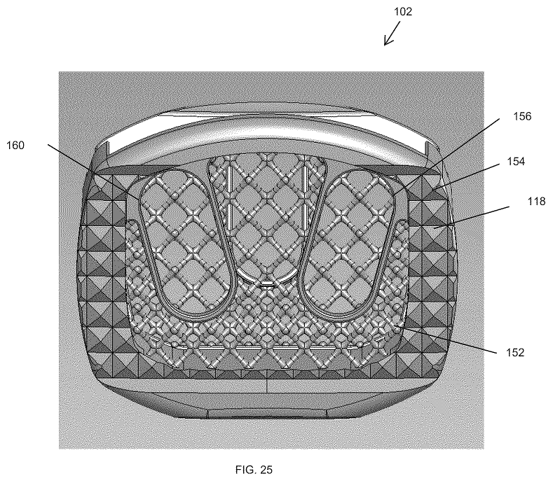

FIG. 25 is a bottom view of the shell of FIG. 1.

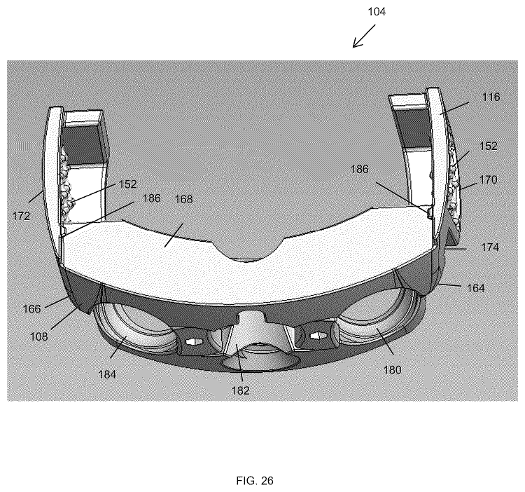

FIG. 26 is a top perspective view of an insert of FIG. 1.

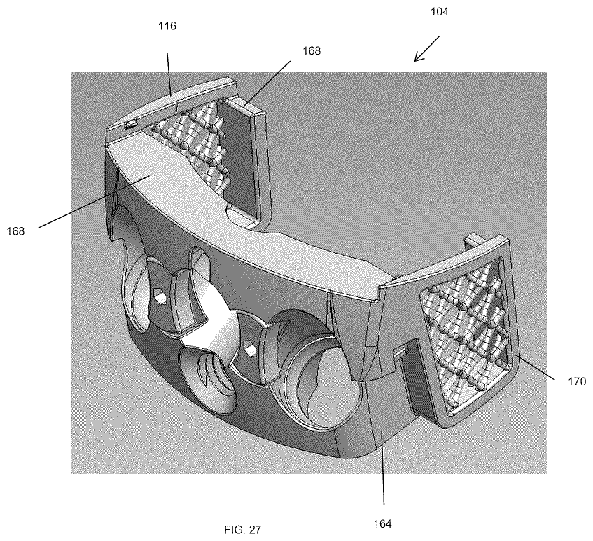

FIG. 27 is another top perspective view of an insert of FIG. 1.

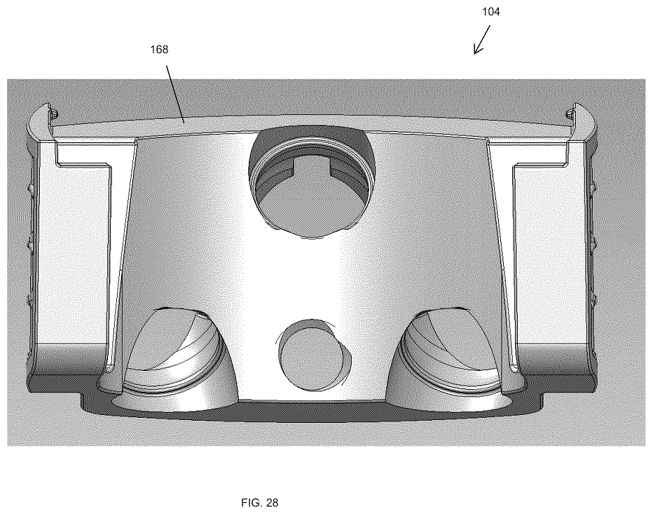

FIG. 28 is a distal view of the insert of FIG. 1.

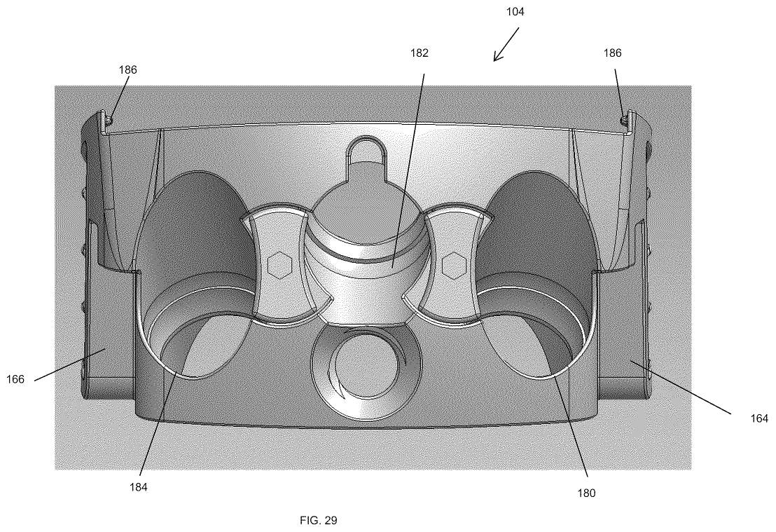

FIG. 29 is a proximal view of the insert of FIG. 1.

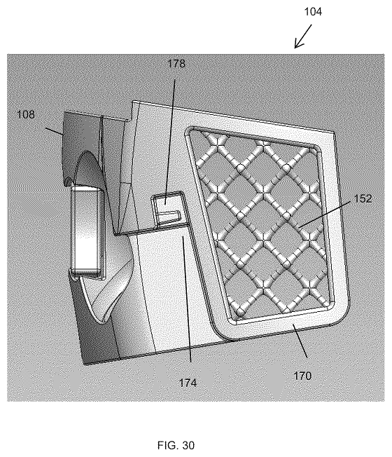

FIG. 30 is a side view of the insert of FIG. 1.

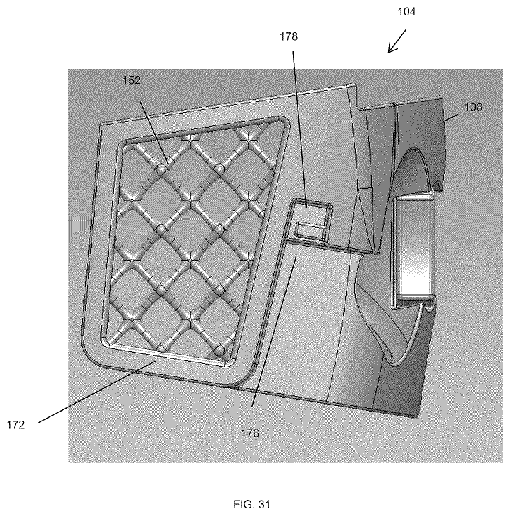

FIG. 31 is another side view of the insert of FIG. 1.

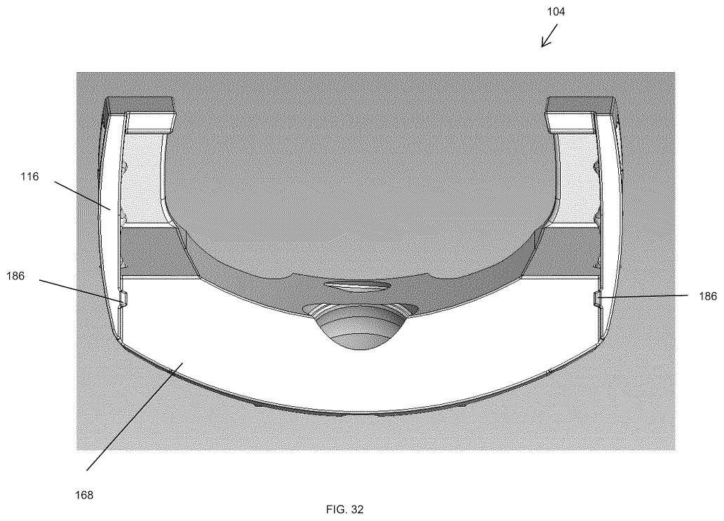

FIG. 32 is a top view of the insert of FIG. 1.

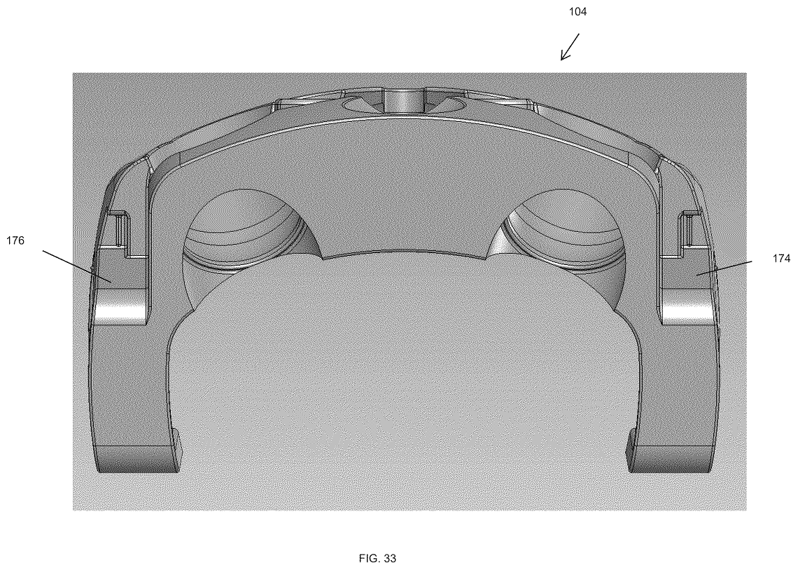

FIG. 33 is a bottom view of the insert of FIG. 1.

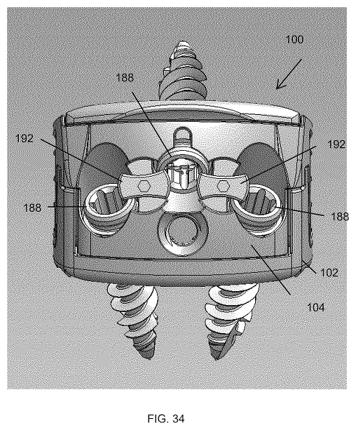

FIG. 34 is a front view of the spinal implant device of FIG. 1 with fasteners.

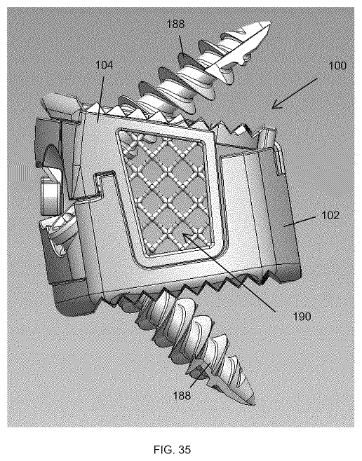

FIG. 35 is a side view of the spinal implant device of FIG. 1 with fasteners.

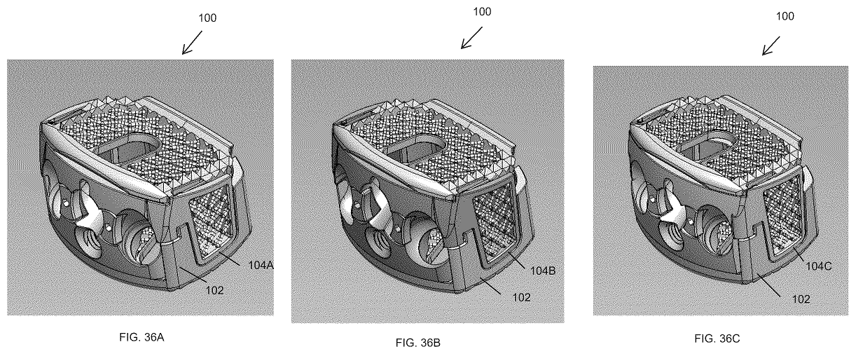

FIGS. 36A-36C are perspective views of embodiments of spinal implant devices with a movable lid shown in a closed position.

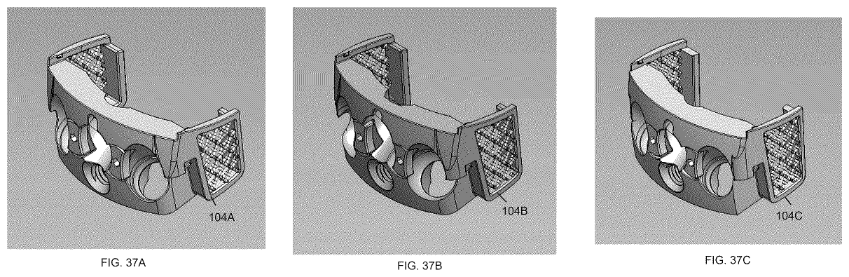

FIGS. 37A-37C are perspective views of embodiments of inserts of the spinal implant devices of FIGS. 36A-36C.

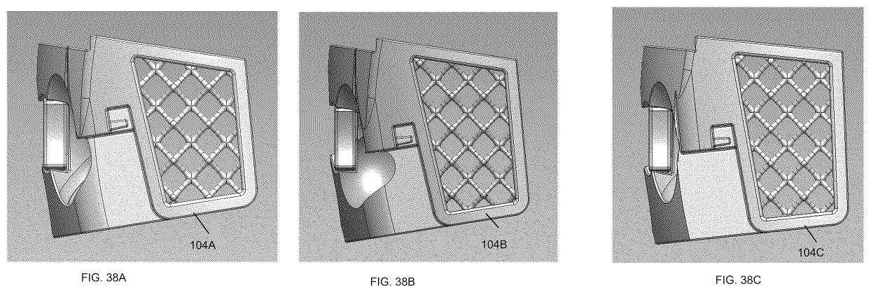

FIGS. 38A-38C are side views of embodiments of inserts of the spinal implant devices of FIGS. 36A-36C.

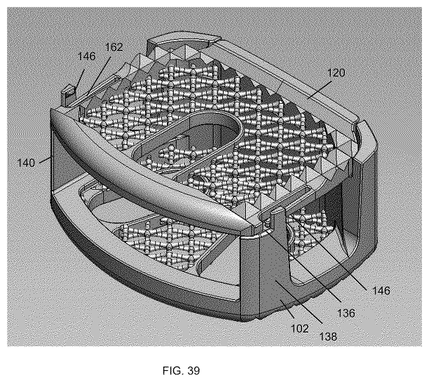

FIG. 39 is a perspective view of an embodiment of a spinal implant device with a movable lid shown in a closed position.

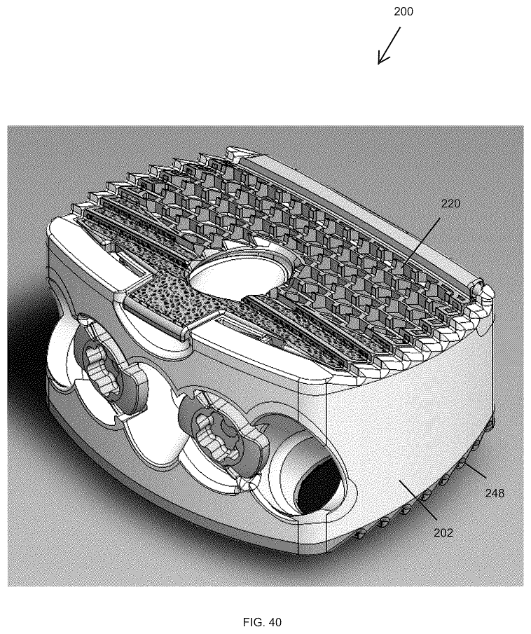

FIG. 40 is a perspective view of an embodiment of a spinal implant device with a movable lid shown in a closed position.

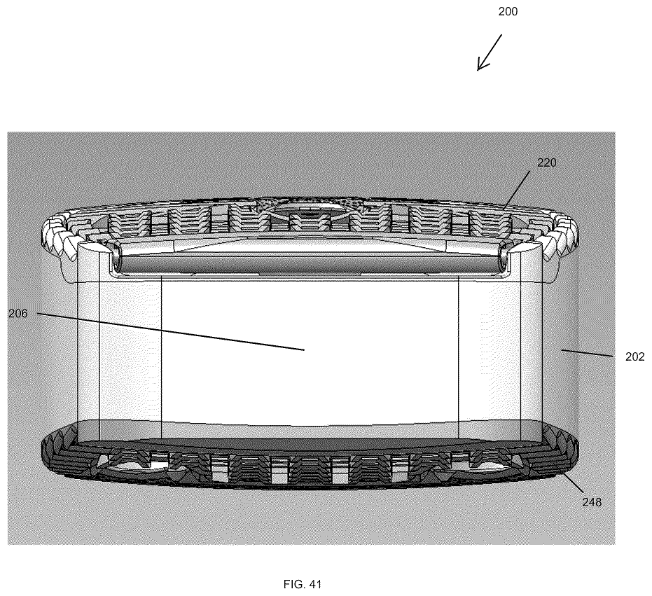

FIG. 41 is a distal view of the spinal implant device of FIG. 40.

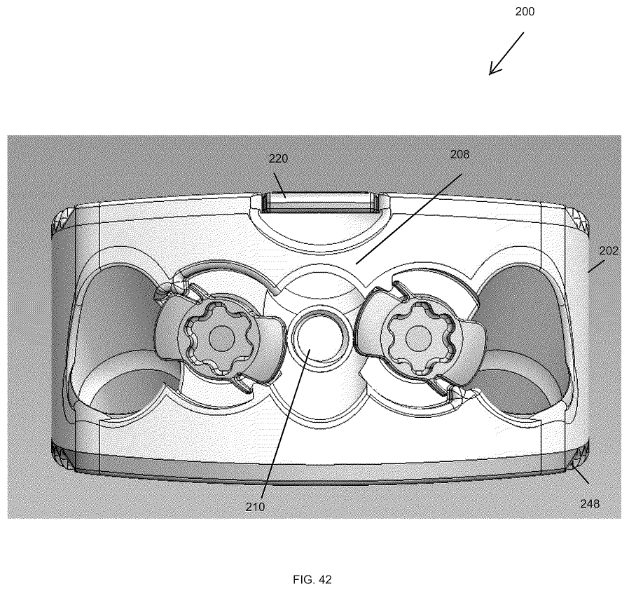

FIG. 42 is a proximal view of the spinal implant device of FIG. 40.

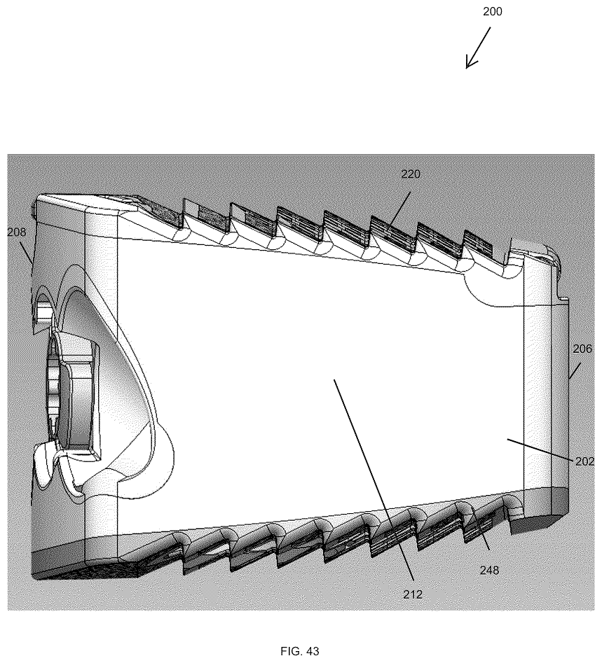

FIG. 43 is a side view of the spinal implant device of FIG. 40.

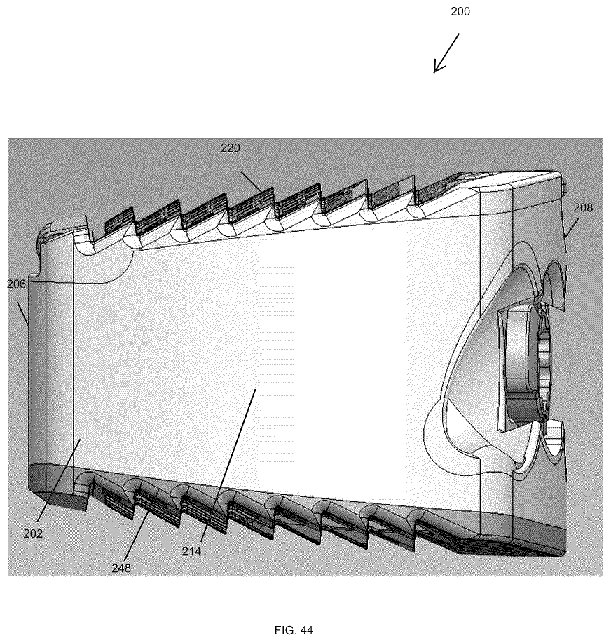

FIG. 44 is another side view of the spinal implant device of FIG. 40.

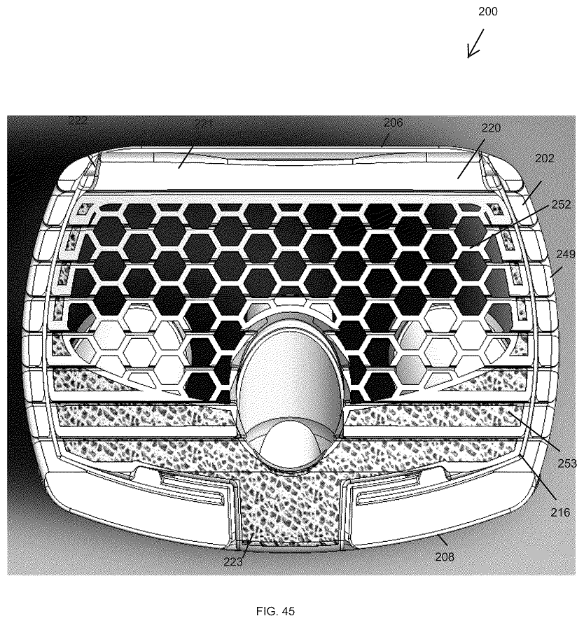

FIG. 45 is a top view of the spinal implant device of FIG. 40.

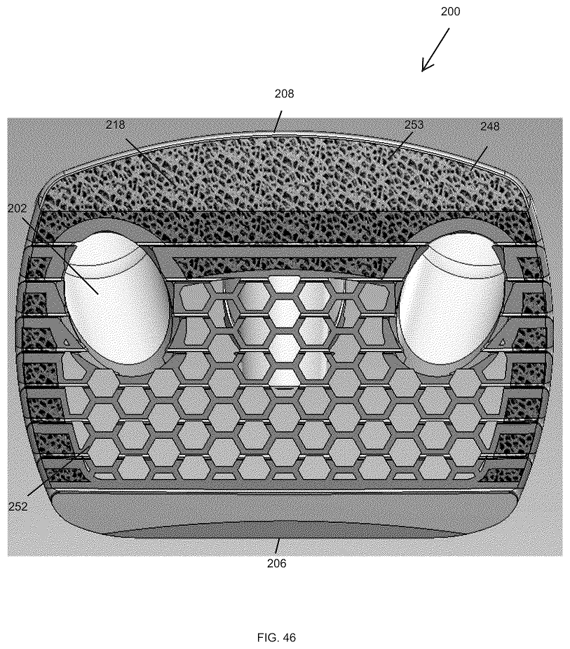

FIG. 46 is a bottom view of the spinal implant device of FIG. 40.

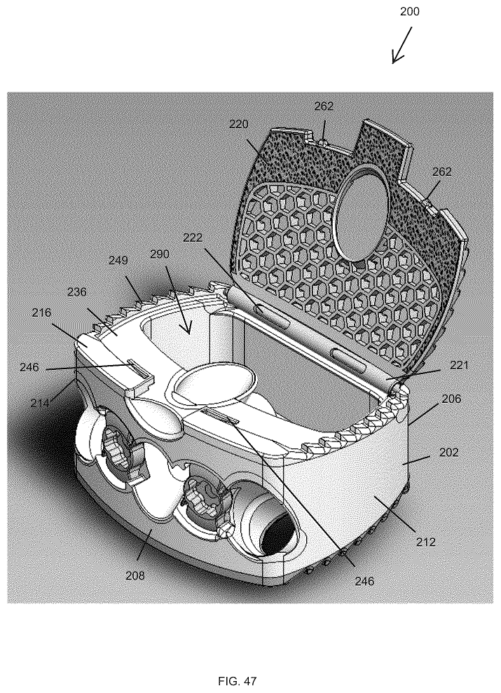

FIG. 47 is a top perspective view of the spinal implant device of FIG. 40 with the movable lid shown in a rotated position.

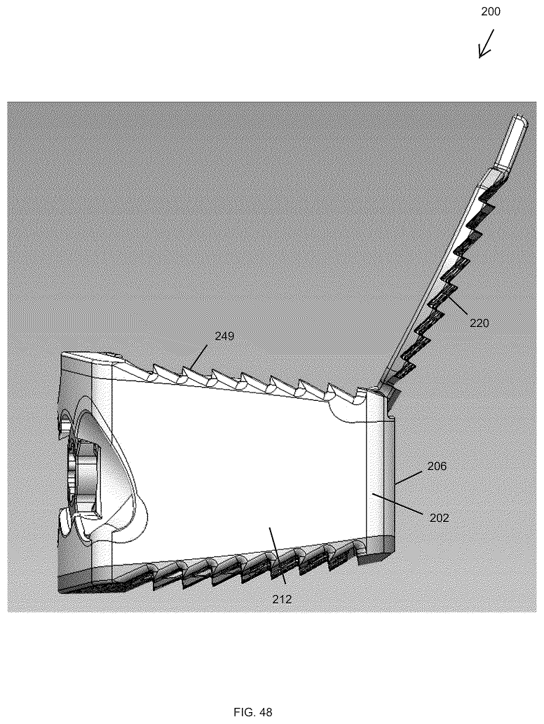

FIG. 48 is a side view of the spinal implant device of FIG. 40 with the movable lid shown in a rotated position.

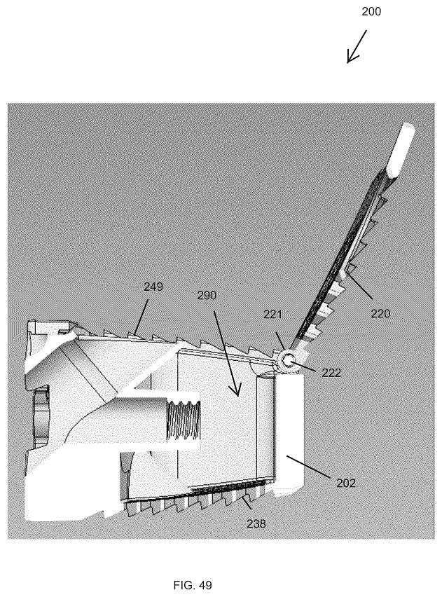

FIG. 49 is a cross-sectional view of the spinal implant device of FIG. 40 with the movable lid shown in a rotated position.

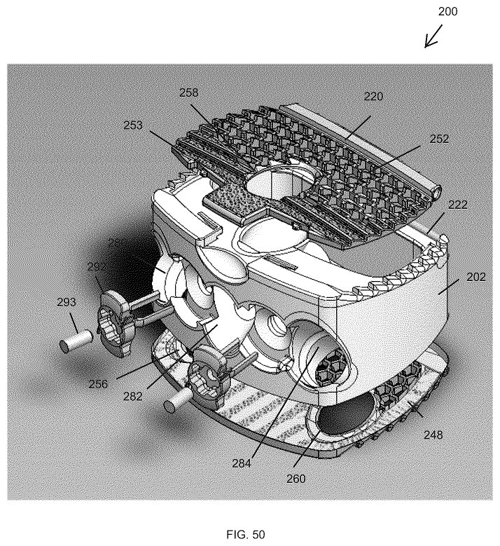

FIG. 50 is an exploded perspective view of the spinal implant device of FIG. 40.

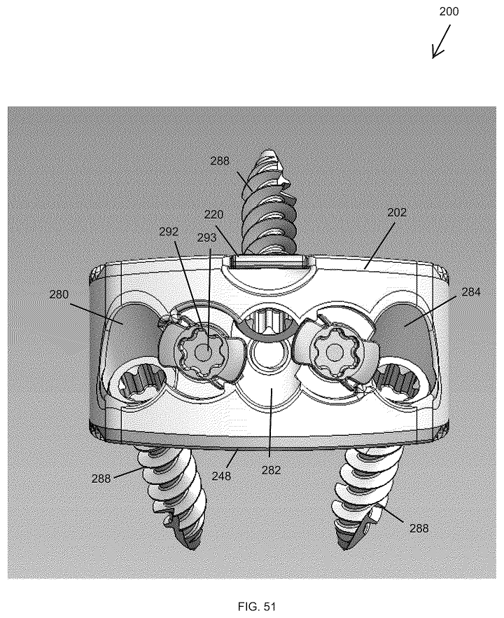

FIG. 51 is a front view of the spinal implant device of FIG. 40 with fasteners.

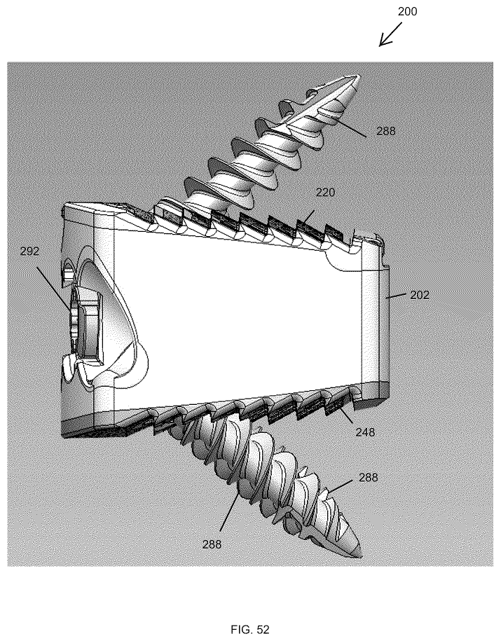

FIG. 52 is a side view of the spinal implant device of FIG. 40 with fasteners.

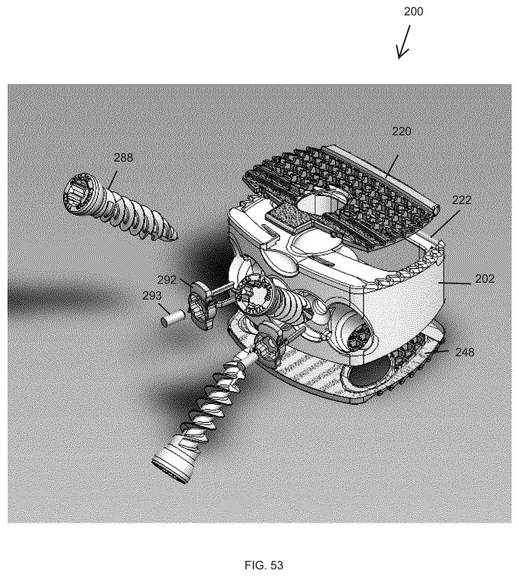

FIG. 53 is an exploded perspective view of the spinal implant device of FIG. 40 with fastener.

### DETAILED DESCRIPTION OF THE PREFERRED EMBODIMENT

FIG. 1 illustrates a perspective view of an embodiment of a spinal implant device 100. The spinal implant device 100 can include a shell 102. The spinal implant device 100 can include an insert 104. The shell 102 and the insert 104 can couple to form the spinal implant device 100 as described herein.

The spinal implant device 100 can be placed between adjacent vertebrae, such as a superior vertebra and an inferior vertebra. The orientation of the spinal implant device 100 between the vertebrae can depend on the insertion direction and the methods of use of the spinal implant device 100. The spinal implant device 100 can be placed at any level of the vertebral column, between any adjacent vertebrae. The spinal implant device 100 can be designed to restore or maintain the spacing between adjacent vertebrae. The spinal implant device 100 can include one or more walls which provide strength and stability to the spinal implant device 100.

FIG. 2 is a distal view of the spinal implant device 100. The spinal implant device 100 can include a distal end 106. In the illustrated embodiment, the shell 102 can form the entire distal end 106. In other embodiments, the shell 102 can form a portion of the distal end 106 and the insert 104 can form a portion of the distal end 106. In some methods of use, the distal end 106 is the leading end which is inserted first into the intervertebral space. In some embodiments, the distal end 106 comprises rounded edges. The distal end 106 can be flat or blunt. The distal end 106 can form any shape for facilitating insertion.

FIG. 3 is a proximal view of the spinal implant device 100. The spinal implant device 100 can include a proximal end 108. The distal end 106 and the proximal end 108 can form opposite ends of the spinal implant device 100. In the illustrated embodiment, the shell 102 can form a portion of the proximal end 108. In the illustrated embodiment, the insert 104 can form a portion of the proximal end 108. In some embodiments, the proximal end 108 comprises rounded edges. The proximal end 108 can be flat or blunt. The proximal end 108 can form any shape. The distance between the distal end 106 and the proximal end 108 can form the length or depth of the spinal implant device 100.

In some embodiments, the length is about 25 mm. In some embodiments, the spinal implant device 100 can have an average length or depth within the range of about 15 mm to about 40 mm (e.g., 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, between 20 and 25 mm, between 25 and 40 mm, between 27 and 37 mm, between 25 and 35 mm, or between 27 and 32 mm, or any range of the foregoing values). In some embodiments, the spinal implant device 100 can include a curve along the length. In some embodiments, the spinal implant device 100 can be straight or substantially straight along the length.

In some methods of use, the proximal end 108 can be configured to be coupled to an insertion tool or driver (not shown). In some embodiments, the proximal end 108 can include an opening 110 to accept the insertion tool. In the illustrated embodiment, the insert 104 can include the opening 110 for the insertion tool. In some embodiments, the opening 110 can be threaded to engage a threaded tip of the insertion tool. In some embodiments, the opening 110 can be adapted to provide an attachment location so that the insertion tool can be connected to the spinal implant device 100. The insertion tool can be used to position the spinal implant device 100 between the vertebrae. The insertion tool can be threaded tightly against the spinal implant device 100. The insertion tool can utilize any connection means including catch/release type mechanism, pin, detent, bayonet connection, or any other connection such that the insertion tool can hold the spinal implant device 100 in a fixed orientation during insertion.

FIG. 4 is a side view of the spinal implant device 100. FIG. 5 is another side view of the spinal implant device 100. The spinal implant device 100 can include two opposing side walls including a first side wall 112 and a second side wall 114. In the illustrated embodiment, the shell 102 can form a portion of the first side wall 112. In the illustrated embodiment, the insert 104 can form a portion of the first side wall 112. In the illustrated embodiment, the shell 102 can form a portion of the second side wall 114. In the illustrated embodiment, the insert 104 can form a portion of the second side wall 114. The side walls 112, 114 can connect the distal end 106 and the proximal end 108.

In some embodiments, each side wall 112, 114 can form a flat surface. In some embodiments, the opposing side walls 112, 114 are separated the same distance along the length of the spinal implant device 100. In some embodiments, the opposing side walls 112, 114 are parallel. In some embodiments, each side wall 112, 114 can form a curved surface. Each side wall 112, 114 can bow outward. Each side wall 112, 114 can be convex. In some embodiments, the opposing side walls 112, 114 are closer near the distal end 106 and farther apart along the length. In some embodiments, the opposing side walls 112, 114 are closer near the proximal end 108 and farther apart along the length. In some embodiments, the opposing side walls 112, 114 are closer near the distal and proximal ends 106, 108 and farther apart near a midpoint along the length. In some embodiments, the first side wall 112 and the second side wall 114 are the same shape, for example generally rectangular. In some embodiments, the first side wall 112 and the second side wall 114 are different shapes. In some embodiments, the distance along the two opposing side walls 112, 114 can form the height of the spinal implant device 100. In some embodiments, the distance between the two opposing side walls 112, 114 can form the width of the spinal implant device 100.

In some embodiments, the spinal implant device 100 has an initial height. In some embodiments, the initial height of the spinal implant device 100 is between 8 mm to 14 mm. In some embodiments, the initial height of the spinal implant device 100 is 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.1 mm, 9.2 mm, 9.3 mm, 9.4 mm, 9.5 mm, 9.6 mm, 9.7 mm, 9.8 mm, 9.9 mm, 10.0 mm, 10.1 mm, 10.2 mm, 10.3 mm, 10.4 mm, 10.5 mm, 10.6 mm, 10.7 mm, 10.8 mm, 10.9 mm, 11.0 mm, 11.1 mm, 11.2 mm, 11.3 mm, 11.4 mm, 11.5 mm, 11.6 mm, 11.7 mm, 11.8 mm, 11.9 mm, 12.0 mm, 12.1 mm, 12.2 mm, 12.3 mm, 12.4 mm, 12.5 mm, 12.6 mm, 12.7 mm, 12.8 mm, 12.9 mm, 13.0 mm, 13.1 mm, 13.2 mm, 13.3 mm, 13.4 mm, 13.5 mm, 13.6 mm, 13.7 mm, 13.8 mm, 13.9 mm, 14.0 mm, between 8.0 mm and 10.0 mm, between 10.0 mm and 12.0 mm, between 12.0 mm and 14.0 mm, or any range of the foregoing values. In some embodiments, the spinal implant device 100 can translate. In some embodiments, the spinal implant device 100 can translate up to an additional 6.0 mm, as described herein. In some embodiments, the spinal implant device 100 can translate 0 mm, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, between 0 mm and 2.0 mm, between 0 mm and 4.0 mm, between 0 mm and 6.0 mm, or any range of the foregoing values.

In some embodiments, the width is about 30 mm. In some embodiments, the spinal implant device 100 can have an average width within the range of about 15 mm to about 40 mm (e.g., 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, between 20 and 25 mm, between 25 and 40 mm, between 27 and 37 mm, between 25 and 35 mm, or between 27 and 32 mm, or any range of the foregoing values).

FIG. 6 is a top view of the spinal implant device 100. FIG. 7 is a bottom view of the spinal implant device 100. The spinal implant device 100 can include two more opposing walls including an upper wall 116 and a lower wall 118. In the illustrated embodiment, the shell 102 can form a portion of the upper wall 116. In the illustrated embodiment, the insert 104 can form a portion of the upper wall 116. In the illustrated embodiment, the shell 102 can form the entire lower wall 118. In the other embodiments, the shell 102 can form a portion of the lower wall 118 and the insert 104 can form a portion of the lower wall 118. The upper wall 116 and the lower wall 118 can connect the distal end 106 and the proximal end 108.

In some embodiments, the upper wall 116 and the lower wall 118 can form a flat surface. In some embodiments, the upper wall 116 and the lower wall 118 are separated the same distance along the length of the spinal implant device 100. In some embodiments, the upper wall 116 and the lower wall 118 are parallel. In some embodiments, the upper wall 116 and the lower wall 118 can form a flat surface. The upper wall 116 and the lower wall 118 can be generally straight. In other embodiments, the upper wall 116 and the lower wall 118 can be curved. In some embodiments, the upper wall 116 and the lower wall 118 are closer near the distal end 106 and farther apart along the length. In some embodiments, the upper wall 116 and the lower wall 118 are closer near the proximal end 108 and farther apart along the length. In some embodiments, the upper wall 116 and the lower wall 118 are closer near the distal and proximal ends 106, 108 and farther apart near a midpoint along the length. In some embodiments, the upper wall 116 and the lower wall 118 are the same shape, for example generally rectangular. In some embodiments, the upper wall 116 and the lower wall 118 are different shapes. In some embodiments, the distance between the upper wall 116 and the lower wall 118 can form the height of the spinal implant device 100. In some embodiments, the distance along the upper wall 116 and the lower wall 118 can form the width of the spinal implant device 100. In some embodiments, the distance along the upper wall 116 and the lower wall 118 can form the length of the spinal implant device 100.

The upper wall 116 and the lower wall 118 can provide load supporting surfaces. In some methods, the upper wall 116 can be positioned adjacent to a vertebral end plate of an upper vertebra. In some methods, the lower wall 118 can be positioned adjacent to a vertebral end plate of a lower vertebra. In some embodiments, the distance between the upper wall 116 and the lower wall 118 corresponds to the height of the intervertebral space where the spinal implant device 100 is to be positioned. In some embodiments, the distance between the upper wall 116 and the lower wall 118 can restore the spacing between adjacent vertebrae. In some methods, when the spinal implant device 100 is positioned between two adjacent vertebrae, the load supporting surfaces of the upper wall 116 and the lower wall 118 contact the vertebral end plates of the adjacent vertebrae. The upper wall 116 and the lower wall 118 are designed to separate the adjacent vertebrae by a distance substantially equal to the total height of the spinal implant device 100.

The spinal implant device 100 can include a movable lid 120. In the illustrated embodiment, the movable lid 120 can be coupled to the distal end 106 of the spinal implant device 100. The moveable lid 120 can be coupled to the spinal implant device 100 at any location to facilitate packing the spinal implant device 100 as described herein. The movable lid 120 is shown in a closed position in FIG. 1.

The movable lid 120 is shown in a rotated position in FIG. 8. FIG. 9 is a side view of the spinal implant device 100 with the movable lid 120 in a rotated position. FIG. 10 is a cross-sectional view of the spinal implant device 100 with the movable lid 120 in a rotated position. The movable lid 120 is shown in a translated position in FIG. 11. FIG. 12 is a side view of the spinal implant device 100 with the movable lid 120 in a translated position. FIG. 13 is a cross-sectional view of the spinal implant device 100 with the movable lid 120 in a translated position. The movable lid 120 is shown in a rotated and translated position in FIG. 14. FIG. 15 is a side view of the spinal implant device 100 with the movable lid 120 in a rotated and translated position. FIG. 16 is a cross-sectional view of the spinal implant device 100 with the movable lid 120 in a rotated and translated position.

In some embodiments, the movable lid 120 can include a double pin joint. The spinal implant device 100 can include a first pin 122. The first pin 122 can extend along the width of the spinal implant device 100, or a portion thereof. The first pin 122 can extend near the distal end 106. The shell 102 can form the first pin 122. The first pin 122 can be integrally or monolithically formed with the shell 102. In other embodiments, the first pin 122 is separately formed from the shell 102. The first pin 122 can be integrally or monolithically formed with the movable lid 120. In other embodiments, the first pin 122 is separately formed from the movable lid 120. The first pin 122 can be cylindrical. The first pin 122 can facilitate rotational movement. The first pin 122 can facilitate translational movement. The first pin 122 can be cylindrical. The first pin 122 can facilitate rotational movement. The first pin 122 can facilitate translational movement.

The spinal implant device 100 can include a second pin 124. The second pin 124 can extend along the width of the spinal implant device 100, or a portion thereof. The second pin 124 can extend near the distal end 106. The shell 102 can form the second pin 124. The second pin 124 can be integrally or monolithically formed with the shell 102. In other embodiments, the second pin 124 is separately formed from the shell 102. The second pin 124 can be elongate. The second pin 124 can facilitate translational movement.

The spinal implant device 100 can include a sleeve 126. The sleeve 126 can be enclosed along a perimeter. The sleeve 126 can include an elongate opening 128. The first pin 122 can be within the elongate opening 128 of the sleeve 126. The first pin 122 can rotate within the elongate opening 128 of the sleeve 126. The first pin 122 can translate within the elongate opening 128 of the sleeve 126. The second pin 124 can be within the elongate opening 128 of the sleeve 126. The sleeve 126 can translate relative to the second pin 124. The sleeve 126 can move in a vertical direction relative to the second pin 124.

The spinal implant device 100 can include a groove 130. In some embodiments, the sleeve 126 can be at least partially within the groove 130. In some embodiments, the sleeve 126 is entirely within the groove 130 in at least one position of the sleeve 126. The sleeve 126 is configured to translate within the groove 130. The sleeve 126 can move in a vertical direction relative to the groove 130.

The movable lid 120 is shown in a rotated position in FIGS. 8-10. The movable lid 120 and the first pin 122 can rotate relative to the sleeve 126. The movable lid 120 and the first pin 122 can rotate relative to the second pin 124. The movable lid 120 and the first pin 122 can rotate relative to the distal end 106. The movable lid 120 and the first pin 122 can rotate relative to the groove 130. The movable lid 120 and the first pin 122 can rotate relative to the side wall 112, 114. The movable lid 120 and the first pin 122 rotate relative to the upper wall 116. The movable lid 120 can rotate, for instance 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, 180°, 185°, 190°, 195°, 200°, 205°, 210°, 215°, 220°, 225°, 230°, 235°, 240°, 245°, 250°, 255°, 260°, 265°, 270°, 275°, 280°, or any range of the foregoing values. In the illustrated embodiment, the movable lid 120 is designed to move in an arc less than 180°.

The movable lid 120 is shown in a translated position in FIGS. 11-13. The movable lid 120, the first pin 122, and the sleeve 126 can translate relative to the second pin 124. The movable lid 120, the first pin 122, and the sleeve 126 can translate relative to the distal end 106. The movable lid 120, the first pin 122, and the sleeve 126 can translate relative to the groove 130. The movable lid 120, the first pin 122, and the sleeve 126 can translate relative to the side wall 112, 114. The movable lid 120, the first pin 122, and the sleeve 126 can translate relative to the upper wall 116. The movable lid 120 can translate, for instance 0 mm, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, or any range of the foregoing values. In some embodiments, the movable lid 100 can designed to translate less than 6.0 mm.

The movable lid 120 is shown in a rotated and translated position in FIGS. 14-16. The movable lid 120 can rotate 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, 180°, between 0° and 90°, between 0° and 180°, up to 180°, or any range of the foregoing values, alone or in combination with translating 0 mm, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, between 0 mm and 6.0 mm, up to 6.0 mm, or any range of the foregoing values. In some embodiments, the movable lid 120 is designed to translate equal to or less than 6.0 mm. In some methods of use, the movable lid 120 rotates, but does not translate. In some methods of use, the movable lid 120 translates, but does not rotate. In some methods of use, the movable lid 120 rotates and translates in combination. The double pin joint permits the movable lid 120 to both rotate between 0° than 180° and translate between 0 mm and 6.0 mm. The double pin joint permits the movable lid 120 to both rotate and translate to house various sized lordosis and various height inserts 104.

The movable lid 120 can comprise a revolute joint, or pin joint, or hinge joint. The first pin 122 functions with one degree of freedom. The movable lid 120 and the first pin 122 rotate. The movable lid 120 and the first pin 122 rotate as a unit. The movable lid 120 and the first pin 122 rotate relative to the distal end 106. The movable lid 120 and the first pin 122 rotate relative to the sleeve 126. The first pin 122 constrains the motion of the movable lid 120 to pure rotation along the axis of the first pin 122. The first pin 122 and the movable lid 120 comprise a cylindrical contact area for rotation. The rotation of the movable lid 120 can be limited by other structures of the spinal implant device 100 such as the side walls 112, 114 or the upper wall 116. The rotation of the movable lid 120 can be limited by other structures of the spinal implant device 100 such as the distal end 106.

The movable lid 120 comprises a translational joint. The first pin 122 functions with one additional degree of freedom. The movable lid 120, the first pin 122, and the sleeve 126 translate. The movable lid 120, the first pin 122, and the sleeve 126 translate as a unit. The second pin 124 constrains the motion of the movable lid 120, the first pin 122, and the sleeve 126 to pure translation along the second pin 124. The second pin 124 comprise an elongated contact area for translation. The translation of the movable lid 120 can be limited by other structures of the spinal implant device 100 such as the length of the elongate opening 128 of the sleeve 126.

The sleeve 126 is configured to translate, but not rotate. The sleeve 126 is configured to translate within the groove 130. The groove 130 guides the vertical motion of the sleeve 126. The sleeve 126 can move in a vertical direction when the first pin 122 and the movable lid 120 move in a vertical direction. The movable lid 120, the first pin 122, and the sleeve 126 can move together. The movable lid 120 can have one rotational degree of freedom and one translational degree of freedom.

FIG. 17 is an exploded perspective view of the spinal implant device 100. The spinal implant device 100 can include the shell 102 and the insert 104. The shell 102 and the insert 104 can include features which facilitate fusion. The shell 102 and the insert 104 can include features which facilitate load transfer. The shell 102 and the insert 104 can include features which facilitate mating.

FIG. 18 is a top perspective view of the shell 102. FIG. 19 is another top perspective view of the shell 102. FIG. 20 is a distal view of the shell 102. FIG. 21 is a proximal view of the shell 102. FIG. 22 is a side view of the shell 102. FIG. 23 is another side view of the shell 102. FIG. 24 is a top view of the shell 102. FIG. 25 is a bottom view of the shell 102. The shell 102 can form the distal end 106. The shell 102 can include the groove 130. The shell 102 can include the sleeve 126. The sleeve 126 can be recessed within the groove 130 such that the distal end 106 can be substantially flat. The shell 102 can include the first pin 122. The shell 102 can include the second pin 124. The shell 102 can include a first distal edge 132 and a second distal edge 134. The groove 130 can be disposed between the first distal edge 132 and the second distal edge 134. The sleeve 126 can be disposed between the first distal edge 132 and the second distal edge 134. The first distal edge 132 and the second distal edge 134 can be rounded.

The shell 102 can include the movable lid 120. The shell 102 can include a ledge 136. The movable lid 120 can be at least partially recessed when contacting the ledge 136. The movable lid 120 can be at least partially recessed when the movable lid 120 is closed. The shell 102 can include the ledge 136 to accommodate the movable lid 120. The ledge 136 can be recessed below the upper wall 116. In some embodiments, the movable lid 120 contacts the ledge 136 when closed. In some embodiments, the movable lid 120 does not contact the ledge 136 when closed. The ledge 136 can extend from the groove 130 along the first distal edge 132. The ledge 136 can extend from the groove 130 along the second distal edge 134. The ledge 136 can be proximal to the groove 130.

The shell 102 can include a first proximal edge 138 and a second proximal edge 140. The first distal edge 132 and the first proximal edge 138 can be connected along a lower surface. The first distal edge 132 and the first proximal edge 138 can be open along an upper surface. The shell 102 can include a first side opening 142 between the first distal edge 132 and the first proximal edge 138. The second distal edge 134 and the second proximal edge 140 can be connected along a lower surface. The second distal edge 134 and the second proximal edge 140 can be open along an upper surface. The shell 102 can include a second side opening 144 between the second distal edge 134 and the second proximal edge 140. The first proximal edge 138 can include the ledge 136. The second proximal edge 140 can include the ledge 136.

The shell 102 can include one or more retention features. The first proximal edge 138 can include a retention feature 146. The second proximal edge 140 can include a retention feature 146. The retention features 146 can be projections. The retention features 146 can project inward. The retention features 146 can be near the proximal end 108. The retention features 146 can be recessed below the upper wall 116. The shell 102 can have an axis of symmetry. The retention features 146 can be mirror image features.

The first proximal edge 138 and the second proximal edge 140 can be connected along a lower surface. The first proximal edge 138 and the second proximal edge 140 can be open along an upper surface. The shell 102 can include a first proximal opening 150 between the first proximal edge 138 and the second proximal edge 140.

The shell 102 can include the lower wall 118. The lower wall 118 can include a porous or network surface 152. The porous or network surfaces 152 can be a matrix. The porous or network surfaces 152 can be square or rectangular. The porous or network surfaces 152 can be planar. The porous or network surfaces 152 can be non-planar. The porous or network surfaces 152 can include rows extending along one plane and alternating rows extending along another plane. The porous or network surfaces 152 can promote bony fusion.

The shell 102 can include the movable lid 120. The movable lid 120 can include the porous or network surface 152. The porous or network surfaces 152 of the lower wall 118 and the movable lid 120 can be vertically aligned. The porous or network surfaces 152 can be on both the upper and lower surfaces of the spinal implant device 100. The porous or network surfaces 152 can be elongate. The porous or network surfaces 152 can extend along the length of the spinal implant device 100, or a portion thereof. The porous or network surfaces 152 can extend along the width of the spinal implant device 100, or a portion thereof. The porous or network surfaces 152 can be parallel. The porous or network surfaces 152 can be aligned to define a vertical flow path between the upper and lower surface of the spinal implant device 100. The porous or network surfaces 152 can be located on opposed surfaces of the spinal implant device 100. In some embodiments, the porous or network surfaces 152 can include different shaped segments. In some embodiments, the porous or network surfaces 152 can include the same perimeter. The spinal implant device 100 can provide access between the adjacent vertebrae. The spinal implant device 100 can provide access through the porous or network surface 152 in the movable lid 120 and the lower wall 118.

The shell 102 can include features to facilitate maintaining the position of the spinal implant device 100 between the vertebrae. The spinal implant device 100 can include a plurality of ridges 154. The ridges 154 can be located on the movable lid 120. The ridges 154 can be located on the lower wall 118. In some embodiments, the ridges 154 are positioned on the upper surface of the spinal implant device 100, the lower surface of the spinal implant device 100, or both the upper surface and the lower surface of the spinal implant device 100.

In some embodiments, the ridges 154 on the upper surface of the spinal implant device 100 and/or the lower surface of the spinal implant device 100 are directionally oriented. The ridges 154 can form a triangular surface sloping upward closer to the proximal end 108. The ridges 154 can be directionally oriented such that the spinal implant device 100 can slide easily in between the vertebrae. The ridges 154 can be directionally oriented such that the spinal implant device 100 can directionally resist being pulled out from between the vertebrae.

The shell 102 can include features to facilitate anchoring the spinal implant device 100. The shell 102 can include zero openings, one opening, two openings, three openings, four openings, five openings, six openings, or any range of the foregoing values. The lower wall 118 can include zero openings, one opening, two openings, three openings, four openings, or any range of the foregoing values. The lower wall 118 can include one or more openings to facilitate anchoring in an inferior vertebra. The movable lid 120 can include zero openings, one opening, two openings, three openings, four openings, or any range of the foregoing values. The movable lid 120 can include one or more openings to facilitate anchoring in a superior vertebra. Two or more openings of the lower wall 118 can be adjacent. Two or more openings of the movable lid 120 can be adjacent. An opening of the lower wall 118 and an opening of movable lid 120 can alternate.

In some embodiments, the shell 102 can include a first opening 156, a second opening 158, and a third opening 160. In some embodiments, the lower wall 118 can include a first opening 156. The movable lid 120 can include a second opening 158. The lower wall 118 can include a third opening 160. In some embodiments, two openings can occur on the lower wall 118 and one opening can occur on the movable lid 120. In some embodiments, the first opening 156, the second opening 158, and the third opening 160 can alternate. In some embodiments, the movable lid 120 can include the first opening 156. The lower wall 118 can include the second opening 158. The movable lid 120 can include the third opening 160. In some embodiments, two openings can occur on the movable lid 120 and one opening can occur on the lower wall 118. In some embodiments, the first opening 156, the second opening 158, and the third opening 160 can alternate. In some embodiments, the shell 102 can include three alternating openings. In some embodiments, the shell 102 can include three openings. The first opening 156, the second opening 158, and the third opening 160 can be spaced apart. The first opening 156, the second opening 158, and the third opening 160 can define separate trajectories through the shell 102. The first opening 156, the second opening 158, and the third opening 160 can have the same shape. The first opening 156, the second opening 158, and the third opening 160 can have different shapes. The first opening 156, the second opening 158, and the third opening 160 can have an elongate opening. The first opening 156, the second opening 158, and the third opening 160 can interrupt the porous or network surface 152. In some embodiments, the lower wall 118 can include one opening (e.g., the first opening 156, or the second opening 158, or the third opening 160). In some embodiments, the lower wall 118 can include two openings (e.g., the first opening 156 and the second opening 158, or the first opening 156 and the third opening 160, or the second opening 158 and the third opening 160). In some embodiments, the movable lid 120 can include one opening (e.g., the first opening 156, or the second opening 158, or the third opening 160). In some embodiments, the movable lid 120 can include two openings (e.g., the first opening 156 and the second opening 158, or the first opening 156 and the third opening 160, or the second opening 158 and the third opening 160).

In some embodiments, the shell 102 can include four openings. In some embodiments, the shell 102 can include the first opening 156, the second opening 158, and the third opening 160, and a fourth opening (not shown). In some embodiments, the lower wall 118 can include two openings (e.g., the first opening 156 and the second opening 158, or the first opening 156 and the third opening 160, or the first opening 156 and the fourth opening, or the second opening 158 and the third opening 160, or the second opening 158 and the fourth opening, or the third opening 160 and the fourth opening). In some embodiments, the movable lid 120 can include two openings (e.g., the first opening 156 and the second opening 158, or the first opening 156 and the third opening 160, or the first opening 156 and the fourth opening, or the second opening 158 and the third opening 160, or the second opening 158 and the fourth opening, or the third opening 160 and the fourth opening). In some embodiments, two openings can occur on the lower wall 118 and two openings can occur on the movable lid 120. In some embodiments, the first opening 156, the second opening 158, the third opening 160, and the fourth opening can alternate. In some embodiments, the movable lid 120 can include the first opening 156 and the third opening 160. In some embodiments, the lower wall 118 can include the second opening 158 and the fourth opening.

The movable lid 120 can include one or more complementary retention features 162. The first side of the movable lid can include the complementary retention feature 162. The second side of the movable lid can include the complementary retention feature 162. The retention feature 146 of the first proximal edge 138 is configured to engage the complementary retention feature 162 of the first side of the movable lid 120, as shown in FIG. 39. The retention feature 146 of the second proximal edge 140 is configured to engage the complementary retention feature 162 of the second side of the movable lid 120. The complementary retention feature 162 flex inward when the movable lid 120 is lowered relative to the first proximal edge 138 and the second proximal edge 140. The complementary retention feature 162 flex inward when contacted with the retention feature 146. The movable lid 120 is further lowered until the complementary retention feature 162 is below the retention feature 146. In some embodiments, the retention feature 146 of the shell 102 is positioned above the complementary retention feature 162 when the movable lid 120 is closed. In some embodiments, the movable lid 120 is positioned against the ledge 136 when the movable lid 120 is closed. The retention features 146 can be configured to retain the movable lid 120 when the movable lid 120 is closed.

FIG. 26 is a top perspective view of the insert 104. FIG. 27 is another top perspective view of the insert 104. FIG. 28 is a distal view of the insert 104. FIG. 29 is a proximal view of the insert 104. FIG. 30 is a side view of the insert 104. FIG. 31 is another side view of the insert 104. FIG. 32 is a top view of the insert 104. FIG. 33 is a bottom view of the insert 104. The insert 104 can form the proximal end 108, or a portion thereof. The insert 104 can include a first proximal edge 164 and a second proximal edge 166. The insert 104 can include the ledge 168. The movable lid 120 can be at least partially recessed when contacting the ledge 168. The movable lid 120 can be at least partially recessed when the movable lid 120 is closed. The insert 104 can include the ledge 168 to accommodate the movable lid 120. The ledge 168 can be recessed below the upper wall 116. The ledge 168 can extend along the first proximal edge 164. The ledge 168 can extend along second proximal edge 166.

The insert 104 can include the first insert side wall 170. As described herein, the shell 102 can include a first side opening 142. The first insert side wall 166 can be disposed within the first side opening 142. The first insert side wall 170 can be coplanar with the shell 102. The insert 104 can include the second insert side wall 172. As described herein, the shell 102 can include a second side opening 144. The second insert side wall 172 can be disposed within the second side opening 144. The second insert side wall 172 can be coplanar with the shell 102.

The first insert side wall 170 can include the porous or network surface 152. The second insert side wall 168 can include the porous or network surface 152. The porous or network surfaces 152 can extend along the height of the spinal implant device 100, or a portion thereof. The porous or network surfaces 152 can be parallel. The porous or network surfaces 152 can be aligned to define a horizontal flow path between the side surfaces of the spinal implant device 100. The porous or network surfaces 152 can be located on opposed surfaces of the spinal implant device 100. In some embodiments, the porous or network surfaces 152 can include different shaped segments. In some embodiments, the porous or network surfaces 152 can include the same perimeter.

The insert 104 can include a first proximal groove 174. The insert 104 can include a second proximal groove 176. As described herein, the shell 102 can include the first proximal edge 138 and the second proximal edge 140. The first proximal groove 174 can receive the first proximal edge 138. The first proximal groove 174 can be lowered relative to the first proximal edge 138. The second proximal groove 176 can receive the second proximal edge 140. The second proximal groove 176 can be lowered relative to the second proximal edge 140. The insert 104 can be lowered relative to the shell 102. As described herein, the first proximal edge 138 can include the retention feature 146 and the second proximal edge 140 can include the retention feature 146. The insert 104 can include complementary insert retention feature 178. The complementary insert retention feature 178 can be a groove 80. The complementary insert retention features 178 of the insert 104 can receive the retention feature 146 of the shell 102.

The insert 104 can include features to facilitate anchoring the spinal implant device 100. The insert 104 can include a first insert opening 180. The insert 104 can include a second insert opening 182. The insert 104 can include a third insert opening 184. As described herein, the shell 102 can include the first opening 156, the second opening 158, and the third opening 160. The first insert opening 180 and the first opening 156 can be aligned along a trajectory for the insertion of a first fastener. The second insert opening 182 and the second opening 158 can be aligned along a trajectory for the insertion of a second fastener. The third insert opening 184 and the third opening 160 can be aligned along a trajectory for the insertion of a third fastener. In some embodiments, the insert 104 can include a fourth insert opening (not shown). The fourth insert opening and the fourth opening of the shell 102 can be aligned along a trajectory for the insertion of a fourth fastener.

The first insert opening 180, the second insert opening 182, and the third insert opening 184 can be aligned. The first insert opening 180, the second insert opening 182, and the third insert opening 184 can be spaced apart. The first insert opening 180, the second insert opening 182, and the third insert opening 184 can define separate trajectories through the insert 104. The first insert opening 180, the second insert opening 182, and the third insert opening 184 can have the same shape. The first insert opening 180, the second insert opening 182, and the third insert opening 184 can have different shapes. The first insert opening 180, the second insert opening 182, and the third insert opening 184 can have an elongate opening.

The insert 104 can include features to facilitate mating with the shell 102. The insert 104 can include one or more retention features 186. The first insert side wall 170 can include the retention feature 186. The second insert side wall 172 can include the retention feature 186. The retention features 186 can be projections. The retention features 186 can be near the proximal end 108. The retention features 186 can be recessed below the upper wall 116. The retention features 186 of the insert 104 and the retention features 146 of the shell 102 can be the same shape. The retention features 186 of the insert 104 and the retention features 146 of the shell 102 can be the same dimensions. The retention features 186 of the insert 104 and the retention features 146 of the shell 102 can have the same function.

Referring back to FIG. 18, the movable lid 120 can include one or more complementary retention features 162. The first side of the movable lid can include the complementary retention feature 162. The second side of the movable lid can include the complementary retention feature 162. The retention feature 186 of the first insert side wall 170 is configured to engage the complementary retention feature 162 of the first side of the movable lid 120, as shown in FIG. 6. The retention feature 186 of the second insert side wall 172 is configured to engage the complementary retention feature 162 of the second side of the movable lid 120. The complementary retention feature 162 flex inward when the movable lid 120 is lowered relative to the first insert side wall 170 and the second insert side wall 172. The complementary retention feature 162 flex inward when contacted with the retention feature 186. The movable lid 120 is further lowered until the complementary retention feature 162 is below the retention feature 186. In some embodiments, the retention feature 186 of the insert 104 is positioned above the complementary retention feature 162 when the movable lid 120 is closed.

In some embodiments, the spinal implant device 100 comprises the shell 102 that is 3D printed as one piece. The movable lid 120 can be 3D printed. The first pin 122 can be 3D printed. The second pin 124 can be 3D printed. The sleeve 126 can be 3D printed. The pins 122, 124 can be captive within the sleeve 126. The sleeve 126 can be captive within the groove 130. In some embodiments, the spinal implant device 100 comprises the insert 104 that is 3D printed as one piece. In some embodiments, the manufacturing method enables the designs described herein. In some embodiments, the spinal implant device 100 would not be able to integrate the movable lid 120 with conventional manufacturing methods.

In some embodiments, the 3D printing process minimizes the material for the shell 102 and the insert 104. In some embodiments, the shell 102 comprises a metal such as titanium. The 3D printing process can minimize the volume of titanium, thus improving radiographic visualization. The 3D printing process can produce the porous or network surface 152. The porous or network surface 152 can be a minimized lattice architecture. The porous or network surface 152 maximizes the flow of graft material. The porous or network surface 152 can improve visualization.

FIG. 34 is a front view of the spinal implant device 100 with fasteners 188. FIG. 35 is a side view of the spinal implant device 100 with fasteners 188. The first insert opening 180 and the first opening 156 can form a trajectory for the insertion of a first fastener 188. The second insert opening 182 and the second opening 158 can form a trajectory for the insertion of a second fastener 188. The third insert opening 184 and the third opening 160 can form a trajectory for the insertion of a third fastener 188.

FIGS. 36A-36C are perspective views of embodiments of spinal implant devices 100. FIGS. 37A-37C are perspective views of embodiments of inserts of the spinal implant devices 100. FIGS. 38A-38C are side views of embodiments of inserts of the spinal implant devices 100. The spinal implant device 100 can include a modular design. The spinal implant device 100 can utilize a universal shell 102. The spinal implant devices 100 can include a single shell 102. The shell 102 can be configured to receive any number of inserts 104 (e.g., zero inserts, one insert, two inserts, three inserts, four inserts, five inserts, six inserts, seven inserts, eight inserts, nine inserts, ten inserts, or any range of the foregoing values). The shell 102 and the plurality of inserts 104 can be a kit. The shell 102 and the plurality of inserts 104 can packaged together. The shell 102 can be configured to receive a plurality of inserts 104A, 104B, 104C. The inserts 104A, 104B, 104C can have any features of the insert 104 described herein. The inserts 104A, 104B, 104C can be smaller than the shell 102. The inserts 104A, 104B, 104C can be smaller in height than the shell 102. The inserts 104A, 104B, 104C can be smaller in length than the shell 102. The inserts 104A, 104B, 104C can be the same width as the shell 102, along at least a portion of the spinal implant device 100. The shell 102 and the plurality of inserts 104A, 104B, 104C can be assembled to produce a plurality of geometries of spinal implant devices 100.

The inserts 104A, 104B, 104C can be engaged by the shell 102 as described herein. The plurality of inserts 104A, 104B, 104C can include retention features 186 that are universal to the plurality of inserts 104. The retention features 186 of the plurality of inserts 104A, 104B, 104C can engage the complementary retention feature 162 of the movable lid 120. The retention features 186 can be universal. The plurality of inserts 104A, 104B, 104C can retain the movable lid 120 of the shell 102 when the movable lid 120 is closed. The shell 102 and the plurality of inserts 104A, 104B, 104C can couple. The retention features 146 of the shell 102 can engage the complementary insert retention feature 178 of the plurality of inserts 104A, 104B, 104C. The plurality of inserts 104A, 104B, 104C can include the first proximal groove 174 and the second proximal groove 176. As described herein, the shell 102 can include the first proximal edge 138 and the second proximal edge 140. The first proximal groove 174 of the plurality of inserts 104A, 104B, 104C can receive the first proximal edge 138 of the shell 102. The first proximal groove 174 of the plurality of inserts 104A, 104B, 104C can be lowered relative to the first proximal edge 138 of the shell 102. The second proximal groove 176 of the plurality of inserts 104A, 104B, 104C can receive the second proximal edge 140 of the shell 102. The second proximal groove 176 of the plurality of inserts 104A, 104B, 104C can be lowered relative to the second proximal edge 140 of the shell 102.

The movable lid 120 of the shell 102 permit compatibility between a single shell 120 and a variety of inserts 104. The movable lid 120 can rotate. The movable lid 120 can translate. The movable lid 120 can have a plurality of closed positions relative to the plurality of inserts 104A, 104B, 104C. The movable lid 120 can be universal. The movable lid 120 can be retained by the plurality of inserts 104A, 104B, 104C. The spinal implant device 100 reduces the number of shells needed for the entire assembled product offering with the plurality of inserts 104A, 104B, 104C.

In some embodiments, the plurality of inserts 104A, 104B comprises different heights. The insert 104A has a shorter height and the insert 104B has a taller height. In some embodiments, the height of the insert 104 is between 6 mm to 13 mm. In some embodiments, the height of the insert 104A is 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.1 mm, 9.2 mm, 9.3 mm, 9.4 mm, 9.5 mm, 9.6 mm, 9.7 mm, 9.8 mm, 9.9 mm, 10.0 mm, 10.1 mm, 10.2 mm, 10.3 mm, 10.4 mm, 10.5 mm, 10.6 mm, 10.7 mm, 10.8 mm, 10.9 mm, 11.0 mm, 11.1 mm, 11.2 mm, 11.3 mm, 11.4 mm, 11.5 mm, 11.6 mm, 11.7 mm, 11.8 mm, 11.9 mm, 12.0 mm, 12.1 mm, 12.2 mm, 12.3 mm, 12.4 mm, 12.5 mm, 12.6 mm, 12.7 mm, 12.8 mm, 12.9 mm, 13.0 mm, between 6.0 mm and 8.0 mm, between 8.0 mm and 10.0 mm, between 11.0 mm and 13.0 mm, or any range of the foregoing values. In some embodiments, the height of the insert 104B is 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.1 mm, 9.2 mm, 9.3 mm, 9.4 mm, 9.5 mm, 9.6 mm, 9.7 mm, 9.8 mm, 9.9 mm, 10.0 mm, 10.1 mm, 10.2 mm, 10.3 mm, 10.4 mm, 10.5 mm, 10.6 mm, 10.7 mm, 10.8 mm, 10.9 mm, 11.0 mm, 11.1 mm, 11.2 mm, 11.3 mm, 11.4 mm, 11.5 mm, 11.6 mm, 11.7 mm, 11.8 mm, 11.9 mm, 12.0 mm, 12.1 mm, 12.2 mm, 12.3 mm, 12.4 mm, 12.5 mm, 12.6 mm, 12.7 mm, 12.8 mm, 12.9 mm, 13.0 mm, between 6.0 mm and 8.0 mm, between 8.0 mm and 10.0 mm, between 11.0 mm and 13.0 mm, or any range of the foregoing values.

In some embodiments, the plurality of inserts 104A, 104C comprises different lordosis. The insert 104A, 104C can be designed with an anatomically oriented lordotic angle. The insert 104A has a greater lordosis and the insert 104C has a smaller lordosis. In some embodiments, the lordosis of the insert 104 is between 0° and 15°. In some embodiments, the lordosis of the insert 104A is 0°, 1°, 2°, 3°, 4°, 5°, 6°, 7°, 8°, 9°, 10°, 11°, 12°, 13°, 14°, 15°, or any range of the foregoing values. In some embodiments, the lordosis of the insert 104B is 0°, 1°, 2°, 3°, 4°, 5°, 6°, 7°, 8°, 9°, 10°, 11°, 12°, 13°, 14°, 15°, or any range of the foregoing values.

FIG. 39 is a perspective view of an embodiment of the spinal implant device 100 with the movable lid 120 shown in a closed position. In some embodiments, the shell 102 can be used without the insert 104. In some embodiments, the shell 102 can be used with a short insert 104. The shell 102 can include one or more retention features 146. The first proximal edge 138 can include the retention feature 146. The second proximal edge 140 can include the retention feature 146. The movable lid 120 can include one or more complementary retention features 162. The first side of the movable lid 120 can include the complementary retention feature 162. The second side of the movable lid 120 can include the complementary retention feature 162. In some embodiments, the one or more retention features 146 of the shell 102 can directly engage the one or more complementary retention feature 162 of the movable lid 120. The shell 102 can retain the movable lid 120 when the movable lid 120 is closed. The shell 102 can include the ledge 136. The movable lid 120 can contact the ledge 136 when the movable lid 120 is closed. The movable lid 120 can be at least partially recessed when the movable lid 120 is closed.

In some embodiments, the spinal implant device 100 can include implant architecture that maximizes graft containment. The shell 102 and the insert 104 can form a central cavity 190. The central cavity 190 can extend the majority of the length of the spinal implant device 100. The central cavity 190 can extend the majority of the width of the spinal implant device 100. The central cavity 190 can extend the majority of the height of the spinal implant device 100. The shell 102 and the insert 104 can maximize the volume of the central cavity 190. The central cavity 190 can be configured to be packed with material.

In some embodiments, the material is graft material. In some embodiments, the graft material can be an autograft, allograft, xenograft or synthetic material. In some embodiments, the graft material is limited to bone graft material which is osteoinductive. In some embodiments, the graft material is limited to bone graft material which is osteogenic. In some embodiments, the synthetic graft material can be ceramic-based, silicon-based or calcium-based. In some embodiments, the graft material can include osteoinductive factors to promote bone ingrowth. In some embodiments, the spinal implant device 100 can be packed with an allogeneic bone scaffold. In some embodiments, the spinal implant device 100 can be packed with allograft or allograft granules. In some embodiments, the spinal implant device 100 can be packed with cancellous or cortical bone. In some embodiments, the spinal implant device 100 can be packed with bone mixed with saline, blood, and/or bone marrow. In some embodiments, the spinal implant device 100 can be packed with demineralized cancellous sponge.

In some embodiments, the spinal implant device 100 comprises the movable lid 120 that encloses the central cavity 190. In some embodiments, the spinal implant device 100 is packed with material before inserting the spinal implant device 100 between vertebrae. The movable lid 120 can retain the graft material during insertion of the spinal implant device 100.

The spinal implant device 100 can include retention features 192. The retention features 192 can retain the head of the fastener 188. In some embodiments, the spinal implant device 100 can include two retention features to retain three fasteners 188. The retention features 192 can rotate. The retention features 192 can have a first position to allow insertion of the fasteners 188 through the insert 104 and the shell 102. The retention features 192 can have a second position to allow retention of the fasteners 188 relative to the insert 104 and the shell 102. The retention features 192 can rotate to transition from the first position to the second position. The retention features 192 can prevent the fasteners 188 from backing out relative to the vertebrae.

The spinal implant device 100 can include a high surface area of the upper and lower surfaces of the spinal implant device 100 that come in contact with the vertebral bodies. This high surface area can reduce the stress on the spinal implant device 100 and the vertebral body, ideally lowering the incidence of subsidence. Subsidence occurs when a spinal implant device migrates into surrounding bone. The movable lid 120 can form the majority of the upper surface of the spinal implant device 100. The increased surface area distributes the load exerted the by vertebra. The large surface area of the movable lid 120 can help to mitigate the risk of subsidence. In some embodiments, the spinal implant device 100 can provide higher surface contact with the endplates, and therefore lower stress to the endplates.

The spinal implant device 100 can be advantageous over conventional devices. Conventional devices can include a cage with relatively thick walls. In some embodiments, the spinal implant device 100 can include one or more thin walls, thus increasing the volume of the central cavity 190. The spinal implant device 100 can include an increase area for graft material. The spinal implant device 100 can allow for more contact with the graft material. The spinal implant device 100 can allow for greater blood infiltration. In some embodiments, the spinal implant device 100 can provide a greater volume of material that can be used in a fusion. In some embodiments, the spinal implant device 100 can deliver a larger volume of material to the disc space. In some embodiments, the spinal implant device 100 can provide better fusion.

The spinal implant device 100 can include one or more shells 102. In some embodiments, the spinal implant device 100 includes only one shell 102. The spinal implant device 100 can include one or more inserts 104. In some embodiments, the spinal implant device 100 includes only one insert 104. In some embodiments, the spinal implant device 100 includes a plurality of different inserts. In some embodiments, the spinal implant device 100 is packaged as a kit with one shell 102 and a plurality of inserts 104. The plurality of inserts 104 can include inserts that couple with a single shell 102. The plurality of inserts 104 can include inserts 104 comprising different heights. The plurality of inserts 104 can include inserts 104 comprising different lordosis. The plurality of inserts 104 can include inserts 104 comprising different footprints. The plurality of inserts 104 can include inserts 104 comprising different materials. In some embodiments, the inserts 104 can be made at least partially of titanium. In other embodiments, the inserts 104 can be made at least partially of polyether ether ketone (i.e., PEEK) or stainless steel.

The spinal implant device 100 can include a modular design. The spinal implant device 100 utilizes an implant assembly wherein the largest component, the shell 102, can engage and house a variety of smaller components, the insert 104, to produce various assembled implant geometries. The plurality of inserts 104 can include retention features 186 that are universal to the plurality of inserts 104. The retention features 186 of the plurality of inserts 104 can engage the complementary retention feature 162 of the movable lid 120. The shell 102 and the plurality of inserts 104 can couple. The plurality of inserts 104 can retain the movable lid 120 of the shell 102 when the movable lid 120 is closed. The moveable aspects of the shell 102 permit compatibility between a single shell 120 and a variety of inserts 104, therefore reducing the number of shell configurations needed for the entire assembled product offering.

The spinal implant device 100 can include a modular concept for insert attachment. The user can choose the desired insert 104. The user can choose the insert 104 from a plurality of inserts 104. The user can choose the insert 104 from two inserts that have different heights. The user can choose the insert 104 from two inserts that have different lordosis.

The spinal implant device 100 can be used with any methods of use described herein. The user can open the movable lid 120. The user can rotate the movable lid 120. The user can translate the movable lid 120. The user can couple the insert 104 to the shell 102. The insert 104 is lowered relative to the shell 102. The shell 102 can include the first proximal edge 138 and the second proximal edge 140. The insert 104 is lowered relative to the first proximal edge 138 and the second proximal edge 140 of the shell 102. The first proximal edge 138 and the second proximal edge 140 can form a keyed surface. The insert 104 can include the first proximal groove 174 and the second proximal groove 176. The first proximal groove 174 and the second proximal groove 176 can form a keyed surface. The first proximal edge 138 can fit within the first proximal groove 174 when the insert 104 is lowered relative to the shell 102. The second proximal edge 140 can fit within the second proximal groove 176 when the insert 104 is lowered relative to the shell 102.

The proximal edge 138 and the second proximal edge 140 of the shell 102 limit or prevent movement of the insert 104 in a lateral direction. The proximal edge 138 and the second proximal edge 140 of the shell 102 limit or prevent movement of the insert 104 relative to the shell 102. The insert 104 can move in a vertical direction relative to the shell 102. The insert 104 is prevented from moving in a horizontal direction relative to the shell 102. The keyed surfaces of the proximal edge 138 and the second proximal edge 140 of the shell 102 and the keyed surfaces of the first proximal groove 174 and the second proximal groove 176 can limit movement between the insert 104 and the shell 102. The proximal edge 138 and the second proximal edge 140 of the shell 102 limit or prevent disengagement of the insert 104 under anatomical loads. The insert 104 can move in a vertical direction relative to the shell 102 for insertion and removal when the movable lid 102 is opened.

The proximal edge 138 and the second proximal edge 140 can include retention features 146 to retain the insert 104. The retention features 146 of the shell 102 can engage the complementary insert retention feature 178 of the insert 104. The retention features 146 of the shell 102 limit or prevent movement of the insert 104 in a vertical direction. The retention features 146 of the shell 102 limit or prevent movement of the insert 104 relative to the shell 102. The insert 104 can move in a vertical direction relative to the shell 102 for insertion and removal by overcoming the retention force of the retention features 146 of the shell 102.

The user can pack the central cavity 190 with graft. The user can pack the central cavity 190 with graft before the spinal implant device 100 is inserted between vertebrae. In other methods, the user can pack the central cavity 190 with graft after the spinal implant device 100 is inserted between vertebrae. The user can close the movable lid 120. The insert 104 can include retention features 186 to retain the movable lid 120 when the movable lid 120 is closed.

The user can insert the spinal implant device 100. The spinal implant device 100 can be inserted between adjacent vertebrae. The spinal implant device 100 can be placed at any level of the vertebral column, between any adjacent vertebrae. The spinal implant device 100 can be configured for insertion between any two vertebrae. The spinal implant device 100 can be configured for insertion between two cervical vertebrae. The spinal implant device 100 can be configured for insertion between two lumbar vertebrae. The spinal implant device 100 can be configured for insertion between two thoracic vertebrae. The spinal implant device 100 can be configured for any approach. The spinal implant device 100 can be configured for an anterior approach. The spinal implant device 100 can be configured for a posterior approach. The spinal implant device 100 can be designed for minimally invasive surgery. The spinal implant device 100 can be designed for Anterior Lumbar Interbody Fusion. The spinal implant device 100 can be designed for Anterior Cervical Discectomy and Fusion. Other placements within the patient are contemplated.

The user can secure the spinal implant device 100 with one or more fasteners 188. The user can insert the first fastener 188 through the first insert opening 180 and the first opening 156, wherein the first insert opening 180 and the first opening 156 are aligned along a trajectory for the insertion of the first fastener 188. The first fastener 188 can be inserted into an inferior vertebra. The user can insert the second fastener 188 through the second insert opening 182 the second opening 158, wherein the second insert opening 182 and the second opening 158 are aligned along a trajectory for the insertion of the second fastener 188. The second fastener 188 can be inserted into a superior vertebra. The user can insert the third fastener 188 through the third insert opening 184 and the third opening 160, wherein the third insert opening 184 and the third opening 160 are aligned along a trajectory for the insertion of the third fastener 188. The third fastener 188 can be inserted into an inferior vertebra. The user can insert the fasteners in any order. In some methods, the first and third fasteners 188 are inserted, and then the second fastener 188 is inserted. The user can insert the first fastener 188 and the third fastener 188 into the inferior vertebra. The user can insert the second fastener 188 into the superior vertebra.

In some embodiments, the user can insert one fastener into the inferior vertebra (e.g., the first fastener 188, or the second fastener 188, or the third fastener 188). In some embodiments, the user can insert one fastener through the lower wall 118 (e.g., the first fastener 188, or the second fastener 188, or the third fastener 188). In some embodiments, the user can insert two fasteners into the inferior vertebra (e.g., the first fastener 188 and the second fastener 188, or the second fastener 188 and the third fastener 188, or the first fastener 188 and the third fastener 188). In some embodiments, the user can insert two fasteners through the lower wall 118 (e.g., the first fastener 188 and the second fastener 188, or the second fastener 188 and the third fastener 188, or the first fastener 188 and the third fastener 188). In some embodiments, the user can insert one fastener into the superior vertebra (e.g., the first fastener 188, or the second fastener 188, or the third fastener 188). In some embodiments, the user can insert one fastener through the movable lid 120 (e.g., the first fastener 188, or the second fastener 188, or the third fastener 188). In some embodiments, the user can insert two fasteners into the superior vertebra (e.g., the first fastener 188 and the second fastener 188, or the second fastener 188 and the third fastener 188, or the first fastener 188 and the third fastener 188). In some embodiments, the user can insert two fasteners through the movable lid 120 (e.g., the first fastener 188 and the second fastener 188, or the second fastener 188 and the third fastener 188, or the first fastener 188 and the third fastener 188). In some embodiments, a single fastener 188 is angled superior. In some embodiments, a single fastener 188 is angled superior and a single fastener 188 is angled inferior. In some embodiments, a single fastener 188 is angled superior and two fasteners 188, 188 are angled inferior. In some embodiments, two fasteners 188, 188 are angled superior and a single fastener 188 is angled inferior. In some embodiments, two fasteners 188 are angled superior and two fasteners 188, 188 are angled inferior.

The spinal implant device 100 can include the shell 102 that can house a variety of inserts 104 and fasteners 188. The shell 102 is capable of both rotational and translational movements, which permit a variety of insert options to determine a final height and lordosis. The shell 102 that permits movement can be 3D printed as one piece. The assembled shell 102 and insert 104 create the central cavity 190 into which material can be packed and enclosed by the movable lid 120. The shell 102 can include a porous or network surface 152 to assist in material enclosure. The porous or network surface can include interruptions for fastener passthrough. The insert 104 can include a porous or network surface 152 to assist in material enclosure.

The spinal implant device 100 can include a double pin joint. In some embodiments, various rigid joints may be used to accomplish the rotational and translational movements. For example, the rotational movement can use a pin joint. The translational joint can use a pin joint, sliding joint, or telescoping linkage. In some embodiments, rigid joints can be replaced by flexible (“compliant”) joints to accomplish the rotational and translational movements. For example, the rotational joint can use a living hinge. The translational joint may use a spring or accordion-like design.

The spinal implant device 100 can include advantages over static implants supplied at discrete height and lordosis options. Static implants cannot be adjusted in geometry, and therefore must be offered in all sizes to cover the full offering. A full static implant offering can be expensive and bulky. The spinal implant device 100 can include the shell 102 that can accept a plurality of inserts 104 having different heights and lordosis options.

The spinal implant device 100 can include advantages over expandable implants that can be adjusted to a final height and/or lordosis in situ. Expandable implants can experience fatigue in the expansion mechanism. Expandable implants require more complex mechanisms to adjust the implant geometry in situ. These complex mechanisms can add cost and impose limits final geometry. The spinal implant device 100 can have modularity to adjust the height and/or lordosis through the selection of the insert 104. This modularity permits a large variety of offerings without the complexity needed for in situ adjustment.

The spinal implant device 100 comprises two components including the shell 102 and the insert 104. The spinal implant device 100 can be less complex and simpler to manufacture than systems with three or more components. The spinal implant device 100 can include components that are coupled to the desired height before insertion. The spinal implant device 100 can include components that prevent disengagement. The spinal implant device 100 can include the first proximal edge 138 and the second proximal edge 140 of the shell 102 that prevent disengagement in a horizontal direction. The spinal implant device 100 can include the first proximal edge 138 and the second proximal edge 140 of the shell 102 that allow mating in a vertical direction, wherein the insert 104 is lowered relative to the shell 102.

The spinal implant device 100 is adjustable in height and lordosis by selecting the insert 104. The insert 104 can be accepted by the shell 102 to form an assembly having the desired height and lordosis before insertion between the vertebrae. The spinal implant device 100 is customizable. The spinal implant device 100 is modular. The user can be provided with a kit that components for assembling an implantable spinal implant device 100. The spinal implant device 100 can be a rigid, non-expandable device. The spinal implant device 100 can enable fusion. The spinal implant device 100 permits the user to configure various implant geometries on the back table, before implantation. This modularity permits a large variety of offerings without the complexity needed for in situ adjustment.

FIG. 40 illustrates a perspective view of an embodiment of a spinal implant device 200. The spinal implant device 200 can include any features of the spinal implant device 100 as described herein.

The spinal implant device 200 can include a body 202. The body 202 can have any features of the shell 102 described herein. The body 202 can have any features of the insert 104 described herein. The spinal implant device 200 can include an endplate 248. The endplate 248 can have any features of the shell 102 described herein. The spinal implant device can include a movable lid 220. The movable lid 220 can have any features of movable lid 120 described herein.

The spinal implant device 200 can be configured to be placed between adjacent vertebrae, such as a superior vertebra and an inferior vertebra. In some embodiments, the movable lid 220 is positioned against the superior vertebra and the endplate 248 is positioned against the inferior vertebra. In some embodiments, the movable lid 220 is positioned against the inferior vertebra and the endplate 248 is positioned against the superior vertebra. The spinal implant device 200 can be placed at any level of the vertebral column, between any adjacent vertebrae. The spinal implant device 200 can be designed to restore or maintain the spacing between adjacent vertebrae. The spinal implant device 200 can be used as an anterior cervical interbody implant. The spinal implant device 200 can be used as a posterior lumbar interbody implant. The spinal implant device 200 can be used as a lateral lumbar interbody implant. The spinal implant device 200 can improve radiographic visualization. The spinal implant device 200 can maximize bone graft volume and containment.

FIG. 41 is a distal view of the spinal implant device 200. The spinal implant device 200 can include a distal end 206. In some embodiments, the body 202 can form a portion of the distal end 206. In some embodiments, the endplate 248 can form a portion of the distal end 206. In some embodiments, the movable lid 220 can form a portion of the distal end 206. In some methods of use, the distal end 206 is the leading end which is inserted first into the intervertebral space. In some embodiments, the distal end 206 can include rounded edges. The distal end 206 can form a flat or blunt surface.

FIG. 42 is a proximal view of the spinal implant device 200. The spinal implant device 200 can include a proximal end 208. In some embodiments, the proximal end 208 can form a curved surface. The proximal end 208 can bow outward. In some embodiments, the body 202 can form a portion of the proximal end 208. In some embodiments, the endplate 248 can form a portion of the proximal end 208. In some embodiments, the movable lid 220 can form a portion of the proximal end 208. In some embodiments, the proximal end 208 can include rounded edges. The proximal end 208 can form a flat or blunt surface. The distance between the distal end 206 and the proximal end 208 can form the length or depth of the spinal implant device 200. The length or depth of the spinal implant device 200 can have any length described herein, including ranges thereof. In some methods of use, the proximal end 208 can be configured to be coupled to an insertion tool or driver (not shown). In some embodiments, the proximal end 208 can include an opening 210 configured to accept the insertion tool. In some embodiments, the body 202 can include the opening 210 for the insertion tool. In some embodiments, the opening 210 can be threaded to engage a threaded tip of the insertion tool.

The spinal implant device 200 can be designed with an anatomically oriented lordotic angle. In some embodiments, the lordosis of the spinal implant device 200 is between 0° and 15°. In some embodiments, the lordosis of the spinal implant device 200 is 0°, 1°, 2°, 3°, 4°, 5°, 6°, 7°, 8°, 9°, 10°, 11°, 12°, 13°, 14°, 15°, or any range of the foregoing values. In some embodiments, the spinal implant device 200 can include a taper from the proximal end 208 toward the distal end 206. In some embodiments, the proximal end 208 has a greater height than the distal end 206. In some embodiments, the spinal implant device 200 can include a taper from the distal end 206 toward the proximal end 208. In some embodiments, the distal end 206 has a greater height than the proximal end 208.

FIG. 43 is a side view of the spinal implant device 200. FIG. 44 is another side view of the spinal implant device 200. The spinal implant device 200 can include two opposing side walls including a first side wall 212 and a second side wall 214. In the illustrated embodiment, the body 202 can form a portion of the first side wall 212. In the illustrated embodiment, the endplate 248 can form a portion of the first side wall 212. In the illustrated embodiment, the movable lid 220 does not form a portion of the first side wall 212. In the illustrated embodiment, the body 202 can form a portion of the second side wall 214. In the illustrated embodiment, the endplate 248 can form a portion of the second side wall 214. In the illustrated embodiment, the movable lid 220 does not form a portion of the second side wall 214. The side walls 212, 214 can connect the distal end 206 and the proximal end 208. In some embodiments, the side walls 212, 214 are solid. In some embodiments, the side walls 212, 214 can include one or more porous or network surfaces as described herein. In some embodiments, each side wall 212, 214 can form a curved surface. Each side wall 212, 214 can bow outward. Each side wall 212, 214 can be convex. In some embodiments, the opposing side walls 212, 214 are closer near the distal end 206 and the proximal end 208 and farther apart near a point along the length. In some embodiments, the opposing side walls 212, 214 are closer near the distal end 206 and the proximal end 208 and farther apart near a midpoint along the length. In some embodiments, the first side wall 212 and the second side wall 214 are the same shape. In some embodiments, the distance along the two opposing side walls 212, 214 can form the height of the spinal implant device 200. The height of the spinal implant device 200 can have any height described herein, including ranges thereof. In some embodiments, the distance between the two opposing side walls 212, 214 can form the width of the spinal implant device 200. The width of the spinal implant device 200 can have any width described herein, including ranges thereof. In some embodiments, the spinal implant device 200 can translate to increase the height as described in relation to spinal implant device 100. In some embodiments, the spinal implant device 200 has a preset height. In some embodiments, the height varies according to the lordotic angle.

FIG. 45 is a top view of the spinal implant device 200. FIG. 46 is a bottom view of the spinal implant device 200. The spinal implant device 200 can include two more opposing walls including an upper wall 216 and a lower wall 218. In the illustrated embodiment, the body 202 can form a portion of the upper wall 216. In the illustrated embodiment, the movable lid 220 can form a portion of the upper wall 216. In the illustrated embodiment, the endplate 248 can form the entire lower wall 218. In some embodiments, the body 202 can form a portion of the lower wall 218.

In some embodiments, the upper wall 216 and the lower wall 218 can form a flat surface. In some embodiments, the upper wall 216 and the lower wall 218 can be separated by a varying height along the length of the spinal implant device 200. In some embodiments, the height varies according to the lordotic angle. In some embodiments, the upper wall 216 and the lower wall 218 are skewed. In some embodiments, the upper wall 216 and the lower wall 218 can form an angle therebetween. In some embodiments, the upper wall 216 and the lower wall 218 are closer near the distal end 206 and farther apart near the proximal end 208. In some embodiments, the upper wall 216 and the lower wall 218 are the same shape. In some embodiments, the distance between the upper wall 216 and the lower wall 218 can form the height of the spinal implant device 200. In some embodiments, the distance along the upper wall 216 and the lower wall 218 can form the width of the spinal implant device 200. In some embodiments, the distance along the upper wall 216 and the lower wall 218 can form the length of the spinal implant device 200.

The upper wall 216 and the lower wall 218 can provide load supporting surfaces. In some methods, the upper wall 216 can be positioned adjacent to the superior vertebra. In some methods, the lower wall 218 can be positioned adjacent to the inferior vertebra. In some methods, when the spinal implant device 200 is positioned between two adjacent vertebrae, the load supporting surfaces of the upper wall 216 and the lower wall 218 contact the adjacent vertebrae. The upper wall 216 and the lower wall 218 are designed to separate the adjacent vertebrae by a distance substantially equal to the height of the spinal implant device 200.

The body 202 can include features to facilitate maintaining the position of the spinal implant device 200 between the vertebrae. The body 202 can form ridges 249 along the side walls 212, 214, or a portion thereof. The ridges 249 can extend from the distal end 206 to the proximal end 208, or a portion thereof. The ridges 249 can form a diagonal tooth. The ridges 249 can prevent retraction of the spinal implant device 200. The body 202 can form ridges 249 over a portion of the body 202. The body 202 can be free of ridges 249 over a portion of the body 202. The proximal end 208 can be free of ridges 249. The body 202 can include one or more solid surfaces.

The movable lid 220 can include one or more porous or network surface 252, 253. The movable lid 220 can include a first porous or network surface 252. The movable lid 220 can include a second porous or network surface 253.

The first porous or network surface 252 and the second porous or network surface 253 can have different features. The first porous or network surface 252 and the second porous or network surface 253 can have different porosity. The first porous or network surface 252 and the second porous or network surface 253 can cover different areas of the movable lid 220. The first porous or network surface 252 and the second porous or network surface 253 can be adjacent in a proximal-distal direction. The first porous or network surface 252 and the second porous or network surface 253 can be adjacent in a lateral direction. The first porous or network surface 252 and the second porous or network surface 253 can perform different functions. The first porous or network surface 252 and the second porous or network surface 253 can have different pore sizes. The first porous or network surface 252 and the second porous or network surface 253 can have different pore shapes. The first porous or network surface 252 and the second porous or network surface 253 can be located in different regions of the movable lid 220. The first porous or network surface 252 and the second porous or network surface 253 can have different load bearing structures. The first porous or network surface 252 can be 3D printed. The second porous or network surface 253 can be 3D printed. The first porous or network surface 252 can be machined. The second porous or network surface 253 can be machined.

The first porous or network surface 252 can be a regional pattern. The first porous or network surface 252 can include polygonal pores. The first porous or network surface 252 can include hexagonal pores. The first porous or network surface 252 can be located on the endplate central structure of the superior surface of the spinal implant device 200. The first porous or network surface 252 can form the same shape as the ridges 249 of the body 202. The first porous or network surface 252 can form a sawtooth. The first porous or network surface 252 can form a diagonal ridge. The first porous or network surface 252 can prevent retraction of the spinal implant device 200. The first porous or network surface 252 can be a matrix. The first porous or network surface 252 can be polygonal. The first porous or network surface 252 can be non-planar. The first porous or network surface 252 can include rows extending along one plane and rows extending along another plane. The first porous or network surface 252 can promote bony fusion by having large open pores.

The first porous or network surface 252 can be advantageous for material containment. The first porous or network surface 252 can be advantageous for retaining graft material within the spinal implant device 200. The first porous or network surface 252 can have maximum pore size. The first porous or network surface 252 can be advantageous for through growth. The first porous or network surfaces 252 can be advantageous for load distribution. The first porous or network surface 252 can have axial strength. The first porous or network surface 252 have a hexagonal pattern. The first porous or network surface 252 have a pattern along a slope. The first porous or network surface 252 can have one side of the polygonal shape along a lower axis and one side of the polygonal shape along an upper axis. The first porous or network surface 252 can have four sides of the polygonal shape along a plane. The plane can be sloped consistent with the ridge 249. The first porous or network surface 252 can form a plurality of ridges that have a shape similar to the ridges 249. The first porous or network surface 252 have a pore size of 1 mm², 1.1 mm², 1.2 mm², 1.3 mm², 1.4 mm², 1.5 mm², 1.6 mm², 1.7 mm², 1.8 mm², 2 mm², 2.1 mm², 2.2 mm², 2.3 mm², 2.4 mm², 2.5 mm², 2.6 mm², 2.7 mm², 2.8 mm², 2.9 mm², 3 mm², 3.1 mm², 3.2 mm², 3.3 mm², 3.4 mm², 3.5 mm², or any range of two of the foregoing values. The first porous or network surface 252 can have a cumulative graft contact area between 50 mm², 55 mm², 60 mm², 65 mm², 70 mm², 75 mm², 80 mm², 85 mm², 90 mm², 95 mm², 100 mm², 105 mm², 110 mm², 115 mm², 120 mm², or any range of two of the foregoing values.

The first porous or network surface 252 can cover a portion of the surface area of the movable lid 220, such as 0%, 5%, 10%, 15%, 20%, 25%, 30%, 35%, 40%, 45%, 50%, 55%, 60%, 65%, 70%, 75%, 80%, 85%, 90%, 100%, more than 30%, more than 50%, less than 70%, less than 50%, or any range of the foregoing values. The first porous or network surface 252 can be located in the middle of the movable lid 220. The first porous or network surface 252 can be spaced inward from ridges 249. The first porous or network surface 252 can be spaced inward from the distal end 206. The first porous or network surface 252 can be spaced inward from the proximal end 208. The first porous or network surface 252 can be spaced inward from lateral sides of the movable lid 202. The first porous or network surface 252 can be spaced inward from all edges of the movable lid 202. The first porous or network surface 252 can be closer to the distal end 206 than the proximal end 208.

The second porous or network surface 253 can be a regional pattern. The second porous or network surface 253 can be a mathematical pattern. The second porous or network surface 253 can include a Voronoi pattern. The second porous or network surface 253 can include a tessellation. The second porous or network surface 253 can be a repeating pattern. The second porous or network surface 253 can be random pattern. The second porous or network surface 253 can have small pores. The second porous or network surface 253 can be located on the endplate edge structure of the superior surface of the spinal implant device 200. The second porous or network surface 253 can be located on the endplate periphery structure. The second porous or network surface 253 can be located along the proximal and side edges of the superior surface.

The second porous or network surface 253 can be advantageous for bone on-growth. The second porous or network surface 253 can have a roughened surface. The second porous or network surface 253 can be advantageous for bone in-growth. The second porous or network surface 253 have a porous surface of 100 μm, 200 μm, 300 μm, 400 μm, 500 μm, 600 μm, 700 μm, 800 μm, 900 μm, 1000 μm, or any range of two of the foregoing values. The second porous or network surface 253 can be 20% porous, 21% porous, 22% porous, 23% porous, 24% porous, 25% porous, 26% porous, 27% porous, 28% porous, 29% porous, 30% porous, 31% porous, 32% porous, 33% porous, 34% porous, 35% porous, 36% porous, 37% porous, 38% porous, 39% porous, 40% porous, or any range of two of the foregoing values. The second porous or network surface 253 can have point spacing of 0.40 mm, 0.42 mm, 0.44 mm, 0.46 mm, 0.48 mm, 0.50 mm, 0.52 mm, 0.54 mm, 0.56 mm, 0.58 mm, 0.60 mm, 0.62 mm, 0.64 mm, 0.66 mm, 0.68 mm, 0.70 mm, or any range of two of the foregoing values. The second porous or network surface 253 can have strut thickness of 0.15 mm, 0.16 mm, 0.17 mm, 0.18 mm, 0.19 mm, 0.20 mm, 0.21 mm, 0.22 mm, 0.23 mm, 0.24 mm, 0.25 mm, 0.26 mm, 0.27 mm, 0.28 mm, 0.29 mm, 0.30 mm, 0.31 mm, 0.32 mm, 0.33 mm, 0.34 mm, 0.35 mm, or any range of two of the foregoing values.

The second porous or network surface 253 can form ridges. The second porous or network surface 253 can be surrounded by solid surfaces. The second porous or network surface 253 can form the same shape as the ridges 249. The second porous or network surfaces 253 can form a sawtooth. The second porous or network surface 253 can form a diagonal ridge. The second porous or network surface 253 can prevent retraction of the spinal implant device 200. The second porous or network surface 253 can form the same shape as the ridges 249 of the body 202. The second porous or network surface 253 can be along a plane. The plane can be sloped consistent with the ridge 249. The second porous or network surface 253 can form a plurality of ridges that have a shape similar to the ridges 249.

The second porous or network surface 253 can cover a portion of the surface area of the movable lid 220, such as 0%, 5%, 10%, 15% 20%, 25%, 30%, 35%, 40%, 45%, 50%, 55%, 60%, 65%, 70%, 75%, 80%, 85%, 90%, 100%, more than 10%, more than 20%, less than 50%, less than 30%, or any range of the foregoing values. The second porous or network surface 253 can be located near the proximal end 208. The second porous or network surface 253 can be located along the proximal edge of the movable lid 220. The second porous or network surface 253 can located along the lateral edges of the movable lid 220. The second porous or network surface 253 can be located along at least three edges of the movable lid 220. The second porous or network surface 253 of the movable lid 220 can at least partially surround the first porous or network surface 252 of the movable lid 220.

The second porous or network surface 253 can have a cumulative graft contact area between 50 mm², 55 mm², 60 mm², 65 mm², 70 mm², 75 mm², 80 mm², 85 mm², 90 mm², 95 mm², 100 mm², 105 mm², 110 mm², 115 mm², 120 mm², or any range of two of the foregoing values. The second porous or network surface 253 can have a smaller cumulative graft contact area than the first porous or network surface 252. The first porous or network surface 252 can contact a greater surface area of the graft material contained within the spinal implant device 200. The second porous or network surface 253 can have smaller pores than the first porous or network surface 252. The second porous or network surface 253 can have less radiolucency than the first porous or network surface 252. The second porous or network surface 253 can be more dense than the first porous or network surface 252. The second porous or network surface 253 can have more material per area than the first porous or network surface 252. The first porous or network surface 252 of the movable lid 220 and the second porous or network surface 253 of the movable lid 220 can comprise the same material. The first porous or network surface 252 of the movable lid 220 and the second porous or network surface 253 of the movable lid 220 can be integrally or monolithically formed. The first porous or network surface 252 of the movable lid 220 and the second porous or network surface 253 of the movable lid 220 can be 3D printed. The first porous or network surface 252 of the movable lid 220 and the second porous or network surface 253 of the movable lid 220 can form a unitary structure. The first porous or network surface 252 of the movable lid 220 and the second porous or network surface 253 of the movable lid 220 can be continuous along the surface of the movable lid 220.

The movable lid 220 can include one or more solid surfaces. The movable lid 220 can include a solid surface near the distal end 206. The movable lid 220 can include a solid surface around the edges of the movable lid 202. The movable lid 220 can include a solid surface near the proximal end 208. The movable lid 220 can include a solid surface surrounding the first porous or network surface 252. The movable lid 220 can include a solid surface surrounding the second porous or network surface 253. The movable lid 220 can include a solid surface surrounding each ridge from by the second porous or network surface 253. The movable lid 220 can include a solid surface along the lower axis of the ridge, a solid surface along the upper axis of the ridge, and a pair of solid surfaces along a plane connecting lower axis an the upper axis. The movable lid 220 can include a solid surface can include four sides surrounding the second porous or network surface 253.

The movable lid 220 can include one or more solid surfaces that supports the function of the movable lid 220. The movable lid 220 can include a solid surface to form a hinge, as described herein. In the illustrated embodiment, the movable lid 220 can be coupled to the distal end 206 of the spinal implant device 200. The movable lid 220 can include a barrel 221 to receive a pin 222 of a hinge joint. The movable lid 220 can include a solid surface that forms the barrel 221. The barrel 221 can be continuous circumferentially. The barrel can be discontinuous circumferentially. The barrel 221 can include a lumen to receive the pin 222. In the illustrated embodiment, the movable lid 220 can pivot relative to the distal end 206. The movable lid 220 can be opened near the proximal end 208. The movable lid 220 can include a tab 223 that facilitates opening and pivoting of the movable lid 220. The tab 223 can include the second porous or network surface 253. The movable lid 220 is shown in a closed position in FIG. 45.

The endplate 248 can include one or more porous or network surface 252, 253. The endplate 248 can include the first porous or network surface 252. The first porous or network surface 252 of the endplate 248 can have any feature described herein. The endplate 248 can include the second porous or network surface 253. The second porous or network surface 253 of the endplate 248 can have any feature described herein. The first porous or network surface 252 can be located on the endplate central structure of the inferior surface of the spinal implant device 200. The first porous or network surface 252 of the endplate 248 can form a mirror image of the shape of the ridges 249. The first porous or network surface 252 can form a hexagonal sawtooth. The first porous or network surface 252 can form a diagonal ridge with a hexagonal profile. The first porous or network surface 252 of the endplate 248 can form a sloped surfaces configured to prevent retraction of the spinal implant device 200.

The first porous or network surface 252 can cover a portion of the surface area of the endplate 248, such as 0%, 5%, 10%, 15% 20%, 25%, 30%, 35%, 40%, 45%, 50%, 55%, 60%, 65%, 70%, 75%, 80%, 85%, 90%, 100%, more than 30%, more than 50%, less than 70%, less than 50%, or any range of the foregoing values. The first porous or network surface 252 can be spaced inward from the distal end 206. The first porous or network surface 252 can be spaced inward from the proximal end 208. The first porous or network surface 252 can be closer to the distal end 206 than the proximal end 208. The first porous or network surface 252 can be spaced inward from the lateral edges of the endplate 248. The first porous or network surface 252 can be encircled by one or more solid surfaces and the second porous or network surface 253.

The second porous or network surface 253 can be located on the endplate edge structure of the inferior surface of the spinal implant device 200. The second porous or network surface 253 can form a mirror image of the shape of the ridges 249. The second porous or network surface 253 can be surrounded by one or more solid surfaces. The second porous or network surface 253 can form ridges surrounded by one or more solid surfaces. The one or more solid surfaces can form the edges of the ridge with the second porous or network surface 253 in the middle. The second porous or network surface 253 can cover a portion of the surface area of the endplate 248, such as 0%, 5%, 10%, 15% 20%, 25%, 30%, 35%, 40%, 45%, 50%, 55%, 60%, 65%, 70%, 75%, 80%, 85%, 90%, 100%, more than 10%, more than 20%, less than 50%, less than 30%, or any range of the foregoing values. The second porous or network surface 253 can be located near the proximal end 208. The second porous or network surface 253 can located along the side edges. The second porous or network surface 253 can at least partially surround the first porous or network surface 252. The endplate 248 can include one or more solid surfaces. The endplate can include a solid surface near the distal end 206.

The first porous or network surface 252 of the movable lid 220 and the first porous or network surface 252 of the endplate 248 can be aligned. The first porous or network surface 252 of the movable lid 220 and the first porous or network surface 252 of the endplate 248 can have the same surface area. The first porous or network surface 252 of the movable lid 220 and the first porous or network surface 252 of the endplate 248 can allow for the vertical ingrowth of bone. The first porous or network surface 252 of the movable lid 220 and the first porous or network surface 252 of the endplate 248 can be located near the center of the superior and inferior surfaces of the spinal implant device 200.

The second porous or network surface 253 of the movable lid 220 and the second porous or network surface 253 of the endplate 248 can be aligned. The second porous or network surface 253 of the movable lid 220 and the second porous or network surface 253 of the endplate 248 can be similar surface area. The second porous or network surface 253 of the movable lid 220 and the second porous or network surface 253 of the endplate 248 can allow for the vertical ingrowth of bone. The second porous or network surface 253 of the movable lid 220 and the second porous or network surface 253 of the endplate 248 can be located along the periphery including the proximal and side edges of the superior and inferior surfaces of the spinal implant device 200.

The first porous or network surface 252 of the endplate 248 and the second porous or network surface 253 of the endplate 248 can comprise the same material. The first porous or network surface 252 of the endplate 248 and the second porous or network surface 253 of the endplate 248 can be integrally or monolithically formed. The first porous or network surface 252 of the endplate 248 and the second porous or network surface 253 of the endplate 248 can be 3D printed. The first porous or network surface 252 of the endplate 248 and the second porous or network surface 253 of the endplate 248 can form a unitary structure. The first porous or network surface 252 of the endplate 248 and the second porous or network surface 253 of the endplate 248 can be continuous along the surface of the endplate 248.

The porous or network surfaces 252, 253 of the movable lid 220 and the endplate 248 can be vertically aligned. The porous or network surfaces 252, 253 can be on both the upper and lower surfaces of the spinal implant device 200. The porous or network surfaces 252, 253 can extend along the length of the spinal implant device 200, or a portion thereof. The porous or network surfaces 252, 253 can extend along the width of the spinal implant device 200, or a portion thereof. The porous or network surfaces 252, 253 can be spaced apart along the height of the spinal implant device 200. The porous or network surfaces 252, 253 can be aligned to define a vertical flow path between the upper and lower surfaces of the spinal implant device 200. The porous or network surfaces 252, 253 can be located on opposed surfaces of the spinal implant device 200. In some embodiments, the first porous or network surface 252 of the movable lid 220 and the first porous or network surface 252 of the endplate 248 can include the same perimeter. In some embodiments, the second porous or network surface 253 of the movable lid 220 and the second porous or network surface 253 have different perimeters. The porous or network surfaces 252, 253 can provide access between the adjacent vertebrae. The porous or network surfaces 252, 253 can provide access in the movable lid 220 and the endplate 248.

The spinal implant device 200 can include a plurality of ridges. The ridges 249 can be formed in the body 202. The ridges can be formed by the first porous or network surface 252 of the movable lid 220. The ridges can be formed by the second porous or network surface 253 of the movable lid 220. The ridges can be formed by one or more solid surfaces of the movable lid 220. The ridges can be formed by the first porous or network surface 252 of the endplate 248. The ridges can be formed by the second porous or network surface 253 of the endplate 248. The ridges can be formed by one or more solid surfaces of the endplate 248. In some embodiments, the ridges are positioned on the upper surface of the spinal implant device 200, the lower surface of the spinal implant device 200, or both the upper surface and the lower surface of the spinal implant device 200. In some embodiments, the ridges on the upper surface of the spinal implant device 200 and/or the lower surface of the spinal implant device 200 are directionally oriented. The ridges can form a triangular surface sloping upward closer to the proximal end 208. The ridges can be directionally oriented such that the spinal implant device 200 can slide easily in between the vertebrae. The ridges can be directionally oriented such that the spinal implant device 200 can directionally resist being pulled out from between the vertebrae.

The movable lid 220 is shown in a closed position in FIGS. 40-46. The movable lid 220 is shown in a rotated position in FIG. 47. FIG. 48 is a side view of the spinal implant device 200 with the movable lid 220 in a rotated position. FIG. 49 is a cross-sectional view of the spinal implant device 200 with the movable lid 220 in a rotated position. The movable lid 220 can rotate relative to the body 202. The movable lid 220 can rotate between an opened position and a closed position.

In some embodiments, the spinal implant device 200 can include the pin 222. The pin 222 can extend along the width of the spinal implant device 200, or a portion thereof. The pin 222 can extend near the distal end 206. In some embodiments, the pin 222 can be integrally or monolithically formed with the body 202. In some embodiments, the pin 222 can be separately formed from the body 202. In some embodiments, the pin 222 can be integrally or monolithically formed with the movable lid 220. In some embodiments, the pin 222 can be separately formed from the movable lid 220. The pin 222 can be cylindrical. The pin 222 can facilitate rotational movement of the movable lid 220. The movable lid 220 can include the barrel 221 to receive the pin 222. The barrel 221 and the pin 222 can form a hinge joint. The movable lid 220 can include a solid surface that forms the barrel 221. The barrel 221 can be continuous circumferentially. The barrel can be discontinuous circumferentially. The barrel 221 can include a lumen to receive the pin 222.

The movable lid 220 can rotate relative to the body 202. The movable lid 220 can rotate relative to the distal end 206. The movable lid 220 can rotate relative to the side wall 212, 214. The movable lid 220 can rotate, for instance 0°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, 180°, 185°, 190°, 195°, 200°, 205°, 210°, 215°, 220°, 225°, 230°, 235°, 240°, 245°, 250°, 255°, 260°, 265°, 270°, 275°, 280°, or any range of the foregoing values. In the illustrated embodiment, the movable lid 220 is designed to move in an arc less than 270°.

The movable lid 220 can comprise a hinge joint. The pin 222 functions with one degree of freedom. The movable lid 220 can rotate about an axis of the pin 222. The rotation of the movable lid 220 can be limited by other structures of the spinal implant device 200 such as the side walls 212, 214 or the upper wall 216. The rotation of the movable lid 220 can be limited by other structures of the spinal implant device 200 such as the distal end 206.

The body 202 can include a ledge 236. The movable lid 220 can be at least partially recessed relative to the body 202 when the movable lid 220 is closed. The body 202 can include the ledge 236 to accommodate the movable lid 220. The ledge 236 can be recessed relative to the upper wall 216. The ledge 236 can be recessed relative to the ridges 249. In some embodiments, the movable lid 220 contacts the ledge 236 when closed. In some embodiments, the movable lid 220 does not contact the ledge 236 when closed.

The body 202 can include one or more retention features 246. The retention feature 246 can be a deflectable arm. The retention feature 246 can project toward the side wall 212. The retention feature 246 can project toward the side wall 214. The retention features 246 can be near the proximal end 208. The retention features 246 can form the upper wall 216. The body 202 can have an axis of symmetry. The retention features 246 can be mirror image features.

The movable lid 220 can include one or more complementary retention features 262. The complementary retention feature 262 can be a projection. The complementary retention feature 262 is configured to engage the retention features 246. The retention feature 246 are deflected when the movable lid 220 is lowered relative to the proximal end 208. The retention features 246 are deflected when contacted with the complementary retention feature 262. The movable lid 220 is further lowered until the complementary retention feature 262 is below the retention feature 246. In some embodiments, the retention feature 246 of the body 202 is positioned above the complementary retention feature 262 of the movable lid 220 when the movable lid 220 is closed. The retention feature 246 can be configured to retain the movable lid 220 when the movable lid 220 is closed.

In some embodiments, the spinal implant device 200 can include implant architecture that maximizes graft containment. The body 202, the movable lid 220, and the endplate 248 can form a central cavity 290. The central cavity 290 can extend the majority of the length of the spinal implant device 200. The central cavity 290 can extend the majority of the width of the spinal implant device 200. The central cavity 290 can extend the majority of the height of the spinal implant device 200. The body 202, the movable lid 220, and the endplate 248 can maximize the volume of the central cavity 290. The central cavity 290 can be configured to be packed with any material described herein. In some embodiments, the spinal implant device 200 is packed with material before inserting the spinal implant device 200 between vertebrae. The movable lid 220 can retain the graft material during insertion of the spinal implant device 200.

In some embodiments, the first porous or network surface 252 of the movable lid 220 overlies the cavity 290. In some embodiments, the first porous or network surface 252 of the endplate 248 overlies the cavity 290. In some embodiments, the second porous or network surface 253 of the movable lid 220 overlies the cavity 290. In some embodiments, the second porous or network surface 253 of the endplate 248 overlies the cavity 290. In some embodiments, the second porous or network surface 253 of the movable lid 220 does not overlie the cavity 290. In some embodiments, the second porous or network surface 253 of the endplate 248 does not overlie the cavity 290. In some embodiments, the second porous or network surface 253 of the movable lid 220 overlies a portion of the body 202. In some embodiments, the second porous or network surface 253 of the endplate 248 overlies a portion of the body 202.

FIG. 50 is an exploded perspective view of the spinal implant device 200. The spinal implant device 200 can include the body 202, the movable lid 220, and the endplate 248. The spinal implant device 200 can include the pin 222. The body 202, the movable lid 220, and the endplate 248 can include features which facilitate fusion. The body 202, the movable lid 220, and the endplate 248 can include features which facilitate load transfer. The body 202, the movable lid 220, and the endplate 248 can include features which facilitate mating.

The body 202 can form the perimeter of the spinal implant device 200. The body 202 can include a first material. The first material can comprise a polymer such as polyetheretherketone (i.e., PEEK). The first material can comprise titanium. The movable lid 220 can form a portion of an upper surface of the spinal implant device 200. The movable lid 220 can comprise the first material. The body 202 and the movable lid 202 can comprise the same material. The movable lid 220 can comprise a second material, different than the first material. The movable lid 220 can comprise a metal such as titanium. The body 202 and the movable lid 220 can comprise different material. The endplate 248 can form the lower surface of the spinal implant device 200. The endplate 248 can comprise the first material. The body 202 and the endplate 248 can comprise the same material. The endplate 248 can comprise the second material. The second material can comprise a metal such as titanium. The endplate 248 and the movable lid 220 can comprise the same material. The endplate can comprise a third material, different than the first material and the second material. The endplate 248 and the body 202 can comprise differential materials. The endplate 248 and the movable lid 220 can comprise different materials. The third material 248 can be a metal such as stainless steel. In some embodiments, the movable lid 220 and the endplate 248 comprise the same material. In some embodiments, the movable lid 220 and the endplate 248 comprise a different material than the body 202.

The spinal implant device 200 can include one or more separate components. The body 202 and the movable lid 220 can be separate components. The body 202 and the endplate 248 can be separate components. The movable lid 220 and the endplate 248 can be separate components. The movable lid 220 can be assembled onto the body 202. The endplate 248 can be assembled onto the body 202. The spinal implant device 200 can pivot about the pin 222. In some embodiments, the movable lid 202 only pivots and does not translate. In some embodiments, the movable lid 202 translates similar to movable lid 102. The spinal implant device 200 can include any features of the spinal implant device 100 described herein.

The spinal implant device 200 can include one or more components that are 3D printed. The body 202 and the endplate 248 can be a printed assembly. The body 202 and the movable lid 220 can be a printed assembly. The body 202, the endplate 248, and the movable lid 220 can be a printed assembly. The body 202 can be removable after printing. The endplate 248 can be removable after printing. The movable lid 220 can be removable after printing. The endplate 248 can be printed separately. The movable lid 220 can be printed separately. The endplate 248 and the movable lid 220 can be assembled to the body 202.

The movable lid 220 can be 3D printed titanium. The body 202 can be 3D printed titanium. The movable lid 220 and the body 202 can be manufactured as a nested print. The movable lid 220 and the body 202 can be printed assembled. The movable lid 220 and the body 202 can be printed to have the pin 222 be captive. In some embodiments, the spinal implant device 200 comprises the body 202 that is 3D printed as one piece. The movable lid 220 can be 3D printed as one piece. The pin 222 can be assembled relative to the movable lid 220 and the body 202.

The movable lid 220 can be 3D printed titanium. The body 202 can be 3D printed PEEK. The movable lid 220 and the body 202 can be manufactured separately. The pin 222 can be manufactured separately. The pin 222 can be press fit to form the assembly. The pin 222 can be a titanium rod. The pin 222 can be inserted through the movable lid 220 and the body 202 to create a hinge. In some embodiments, the movable lid 220 pivots about the pin 222. In some embodiments, the movable lid 220 and the body 202 are coupled via a hinge. In some embodiments, the movable lid 220 and the body 202 are coupled via a snap fit.

In some embodiments, a method of manufacturing is provided. The movable lid 220 can be 3D printed titanium. The body 202 can be 3D printed titanium. The movable lid 220 and the body 202 can be manufactured as a nested print. The movable lid 220 and the body 202 can be manufactured as a printed assembly. In some embodiments, the pin 222 can be 3D printed titanium. In some embodiments, the pin 222 can be captive within the movable lid 220.

In some embodiments, a method of manufacturing is provided. The movable lid 220 can be 3D printed titanium. The body 202 can be 3D printed PEEK. The movable lid 220 and the body 202 can not be manufactured as a nested print. The movable lid 220 and the body 202 can not be manufactured as a printed assembly. In some embodiments, the pin 222 is 3D printed. In some embodiments, the pin 222 is 3D printed titanium. In some embodiments, the pin 222 is 3D printed with the movable lid 220. In some embodiments, the pin 222 is 3D printed separately from the movable lid 220. In some embodiments, the pin 222 can be a titanium rod. In some embodiments, the pin 222 can be assembled to the body 202. The pin 222 can be press fit through the movable lid 220 and the body 202. The pin 222 can be press fit to create the hinge. In some embodiments, the spinal implant device 200 uses a different mechanism than a hinge. In some embodiments, the spinal implant device 200 uses a snap fit between the movable lid 220 and the body 202. In some embodiments, the top periphery of the spinal implant device 200 comprises PEEK. In some embodiments, the upper wall 216 of the spinal implant device 200 comprises both PEEK and titanium.

In some embodiments, a method of manufacturing is provided. The movable lid 220 can be 3D printed titanium. The endplate 248 can be 3D printed titanium. The body 202 can be 3D printed PEEK. The endplate 248 can be constrained in multiple locations. In some embodiments, the endplate 248 can be constrained with double cross-pins, one posterior and one anterior. In some embodiments, the endplate 248 can be constrained with a different attachment mechanism. In some embodiments, the endplate 248 can be constrained with a snap fit. In some embodiments, the endplate 248 can be constrained with a dovetail.

In some embodiments, the 3D printing process minimizes the material for the body 202, the movable lid 220, and the endplate 248. The 3D printing process can minimize the volume of material used for each component, thus improving radiographic visualization. The 3D printing process can produce the porous or network surfaces 252, 253. The porous or network surfaces 252, 253 can be a minimized lattice architecture. The porous or network surfaces 252, 253 can maximize the flow of graft material. The porous or network surfaces 252, 253 can improve visualization.

In some embodiments, referring to FIGS. 45 and 50, the body 202 forms a portion of the upper wall 216. The body 202 can be positioned laterally relative to the movable lid 220. The body 202 can form one or more edges of the upper wall 216. The body 202 can form the top periphery. The upper wall 216 can include two materials such as PEEK of the body 202 and titanium of the movable lid 220. The upper wall 216 can include a portion of the body 202 formed from the first material and the movable lid 220 formed from the second, different material.

The endplate 248 can include features to facilitate mating with the body 202. The endplate 248 can include coupling features. The proximal end 208 can include one or more complementary coupling features. The distal end 206 can include one or more complementary coupling features. The side wall 212, 214 can include one or more complementary coupling features. The endplate 248 can be coupled to the body 202 along the periphery of the spinal implant device 200. The endplate 248 can be coupled to the body 202 at multiple locations. The endplate 248 can be coupled to the body 202 using one or more cross-pins. The endplate 248 can be coupled to the body 202 using a double cross-pin. The endplate 248 can be coupled to the body 202 using one or more pins that couple the endplate 248 and the proximal end 208. The endplate 248 can be coupled to the body 202 using one or more pins that couple the endplate 248 and the distal end 206. The endplate 248 can be coupled to the body 202 with a snap fit. The endplate 248 can be coupled to the body 202 with a dovetail. The endplate 248 can be coupled to the body 202 with one or more fasteners such as screws or rivets. The endplate 248 can be coupled to the body 202 with an adhesive. The endplate 248 can be coupled to the body 202 with a flange. The endplate 248 can be coupled to the body 202 through welding. The endplate 248 can be coupled to the body 202 through overmolding.

The body 202 can include features to facilitate anchoring the spinal implant device 200. The body 202 can include zero openings, one opening, two openings, three openings, four openings, five openings, six openings, or any range of the foregoing values. The endplate 248 can include zero openings, one opening, two openings, three openings, four openings, or any range of the foregoing values. The endplate 248 can include one or more openings to facilitate anchoring in the inferior vertebra. The movable lid 220 can include zero openings, one opening, two openings, three openings, four openings, or any range of the foregoing values. The movable lid 220 can include one or more openings to facilitate anchoring in the superior vertebra. The openings of the endplate 248 and the openings of movable lid 220 can alternate.

In some embodiments, the spinal implant device 200 can include a first opening 256, a second opening 258, and a third opening 260. In some embodiments, the endplate 248 can include the first opening 256. The movable lid 220 can include the second opening 258. The endplate 248 can include the third opening 260. The first opening 256 and the third opening 260 of the endplate 248 can interrupt the first porous or network surface 252 of the endplate 248. The first opening 256 and the third opening 260 of the endplate 248 can interrupt the second porous or network surface 253 the endplate 248. The second opening 258 of the movable lid 220 can interrupt the first porous or network surface 252 of the movable lid 220. The second opening 258 of the movable lid 220 can interrupt the second porous or network surface 253 the movable lid 220. The first opening 256, the second opening 258, and the third opening 260 can interrupt the porous or network surfaces 252, 253. In some embodiments, two openings can occur on the endplate 248 and one opening can occur on the movable lid 220. In some embodiments, the first opening 256, the second opening 258, and the third opening 260 can alternate. In some embodiments, the movable lid 220 can include the first opening 256. The endplate 248 can include the second opening 258. The movable lid 220 can include the third opening 260. The spinal implant device 200 can include three alternating openings. The first opening 256, the second opening 258, and the third opening 260 can define separate trajectories through the spinal implant device 200.

The body 202 can include features to facilitate anchoring the spinal implant device 200. The body 202 can include a first body opening 280. The body 202 can include a second body opening 282. The body 202 can include a third body opening 284. As described herein, the moveable lid 202 and the endplate 248 can include the first opening 256, the second opening 258, and the third opening 260. The first body opening 280 and the first opening 256 of the endplate 248 can be aligned along a trajectory for the insertion of a first fastener. The first body opening 280 can form an enclosed lumen toward the first opening 256 of the endplate 248. The second body opening 282 and the second opening 258 of the movable lid 220 can be aligned along a trajectory for the insertion of a second fastener. The second body opening 282 can form an enclosed lumen toward the second opening 258 of the movable lid 220. The third body opening 284 and the third opening 260 of the endplate 248 can be aligned along a trajectory for the insertion of a third fastener. The third body opening 284 can form an enclosed lumen toward the third opening 260 of the endplate 248. The body 202 can form a solid surface that encompasses the first body opening 280, the second body opening 282 and the third body opening 284.

The first body opening 280, the second body opening 282, and the third body opening 284 can be aligned. The first body opening 280, the second body opening 282, and the third body opening 284 can be spaced apart. The first body opening 280, the second body opening 282, and the third body opening 284 can define separate trajectories. The first body opening 280, the second body opening 282, and the third body opening 284 can have the same shape. The first body opening 280, the second body opening 282, and the third body opening 284 can have different shapes. The first body opening 280, the second body opening 282, and the third body opening 284 can have an elongate opening.

FIG. 51 is a front view of the spinal implant device 200 with fasteners 288. FIG. 52 is a side view of the spinal implant device 200 with fasteners 288. FIG. 53 is an exploded view of the spinal implant device 200 with fasteners 288. The first body opening 280 and the first opening 256 of the endplate 248 can form a trajectory for the insertion of a first fastener 288. The second body opening 282 and the second opening 258 of the movable lid 220 can form a trajectory for the insertion of a second fastener 288. The third insert opening 284 and the third opening 260 can form a trajectory for the insertion of a third fastener 288.

The spinal implant device 200 can include retention features 292. The retention features 292 can retain the head of the fastener 288. In some embodiments, the spinal implant device 200 can include two retention features 292 to retain three fasteners 288. The retention features 292 can rotate. The retention features 292 can have a first position to allow insertion of the fasteners 288 through the body 202. The retention features 292 can have a second position to allow retention of the fasteners 288 relative to the body 202. The retention features 292 can rotate to transition from the first position to the second position. The retention features 292 can prevent the fasteners 288 from backing out relative to the vertebrae. The retention feature 292 can rotate relative to a pin 293. The pin 293 can couple the retention feature 292 to the body 202. The retention feature 292 and the body openings 280, 282, 284 can alternate.

The spinal implant device 200 can include one or more surfaces with porosity patterns. The patterns can be hexagonal and Voronoi. The spinal implant device 200 can include a combination of two or more porous or network surfaces 252, 253 on the movable lid 220. The spinal implant device 200 can include a combination of two or more porous or network surfaces 252, 253 on the endplate 248. The spinal implant device 200 can include a combination of two or more porous or network surfaces 252, 253 on different surfaces. The spinal implant device 200 can include a combination of two or more porous or network surfaces 252, 253 to improve radiolucency. The spinal implant device 200 can include a combination of two or more porous or network surfaces to improve graft retention. The spinal implant device 200 can include a combination of two or more porous or network surfaces 252, 253 that are 3D printed. The spinal implant device 200 can include a combination of two or more porous or network surfaces 252, 253 that are integrally formed on the movable lid 220. The spinal implant device 200 can include a combination of two or more porous or network surfaces 252, 253 that are integrally formed on the endplate 248.

The spinal implant device 200 can include a high surface area of the upper and lower surfaces of the spinal implant device 200 that come in contact with the vertebral bodies. This high surface area can reduce the stress on the spinal implant device 200 and the vertebral body, ideally lowering the incidence of subsidence. The movable lid 220 can form the majority of the upper surface of the spinal implant device 200. The endplate 248 can form the entire lower surface of the spinal implant device 200. The movable lid 220 and the endplate 248 distribute the load exerted by vertebrae. The large surface area of the movable lid 220 and the endplate 248 can help to mitigate the risk of subsidence. In some embodiments, the movable lid 220 and the endplate 248 can provide higher surface contact with the endplates of the vertebrae, and therefore lower stress to the endplates of the vertebrae.

The spinal implant device 200 can be used with any methods of use described herein. The user can open the movable lid 220. The user can rotate the movable lid 220. The user can pack the central cavity 290 with graft. The user can pack the central cavity 290 with graft before the spinal implant device 200 is inserted between vertebrae. In other methods, the user can pack the central cavity 290 with graft after the spinal implant device 200 is inserted between vertebrae. The user can close the movable lid 220. The user can insert the spinal implant device 200. The spinal implant device 200 can be inserted between adjacent vertebrae. The user can secure the spinal implant device 200 with one or more fasteners 288. The user can rotate the one or more retention features 292 to prevent backout of the fasteners 288. The user can complete any method step described herein.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims. Although the present invention has been described in relation to various exemplary embodiments, various additional embodiments and alterations to the described embodiments are contemplated within the scope of the invention. Thus, no part of the foregoing description should be interpreted to limit the scope of the invention as set forth in the following claims. For all of the embodiments described above, the steps of the methods need not be performed sequentially.

1. A spinal implant device comprising:
a shell comprising:
a distal end and a proximal end,
a lower wall forming a lower surface of the shell;
a movable lid forming an upper surface of the shell, wherein the movable lid is configured to rotate and translate; and
an insert configured to couple with the shell.

2. The spinal implant device of claim 1, wherein the movable lid is configured to rotate up to 180 degrees.

3. The spinal implant device of claim 1, wherein the movable lid is configured to translate between 0 mm and 6 mm.

4. (canceled)

5. (canceled)

6. (canceled)

7. (canceled)

8. (canceled)

9. (canceled)

10. The spinal implant device of claim 1, wherein the insert comprises a retention feature and the movable lid comprises a complementary retention feature.

11. The spinal implant device of claim 1, wherein the insert comprises a first insert opening and the lower wall comprises a first opening, wherein the first insert opening and the first opening are aligned along a trajectory for the insertion of a first fastener.

12. (canceled)

13. (canceled)

14. The spinal implant device of claim 1, wherein the insert comprises a first insert opening and the movable lid comprises a first opening, wherein the first insert opening and the first opening are aligned along a trajectory for the insertion of a first fastener.

15. (canceled)

16. (canceled)

17. (canceled)

18. (canceled)

19. (canceled)

20. A method comprising:
providing a shell comprising:
a distal end and a proximal end,
a lower wall forming a lower surface of the shell;
a movable lid;
opening the movable lid, wherein the movable lid rotates and translates; and
lowering an insert toward the lower wall to couple with the shell.

21. (canceled)

22. (canceled)

23. (canceled)

24. A spinal implant device comprising:
a body;
a movable lid configured to rotate relative to the body;
an endplate, wherein the body, the movable lid, and the endplate form a cavity configured to be packed with material.

25. The spinal implant device of claim 24, further comprising a pin, wherein the movable lid is configured to rotate relative to the pin.

26. The spinal implant device of claim 25, wherein the pin comprises titanium.

27. The spinal implant device of claim 24, wherein the movable lid and the endplate are separate components configured to be assembled onto the body.

28. The spinal implant device of claim 24, wherein the movable lid is printed as a separate piece.

29. The spinal implant device of claim 24, wherein the movable lid and the body comprise a printed assembly.

30. The spinal implant device of claim 24, wherein the endplate is printed as a separate piece.

31. The spinal implant device of claim 24, wherein the endplate and the body comprise a printed assembly.

32. The spinal implant device of claim 24, wherein the endplate and the body are coupled with one or more pins.

33. The spinal implant device of claim 24, wherein the movable lid comprises titanium.

34. The spinal implant device of claim 24, wherein the body comprises PEEK.

35. The spinal implant device of claim 24, wherein the endplate comprises titanium.

36. The spinal implant device of claim 24, wherein an upper wall comprises titanium and PEEK.
